SEQUENCE LISTING (25 sequences)

SEQ ID NO: 1 - protein, 1144 aa, Homo sapiens
MKLSRQFTVFGSAIFCVVIFSLYLMLDRGHLDYPRNPRREGSFPQGQLSMLQEKIDHLERLLAENNEIISNIRDSVINLSESVEDGPKSSQSNFSQGAGSHLLPSQLSLSVDTADCLFASQSGSHNSDVQMLDVYSLISFDNPDGGVWKQGFDITYESNEWDTEPLQVFVVPHSHNDPGWLKTFNDYFRDKTQYIFNNMVLKLKEDSRRKFIWSEISYLSKWWDIIDIQKKDAVKSLIENGQLEIVTGGWVMPDEATPHYFALIDQLIEGHQWLENNIGVKPRSGWAIDPFGHSPTMAYLLNRAGLSHMLIQRVHYAVKKHFALHKTLEFFWRQNWDLGSVTDILCHMMPFYSYDIPHTCGPDPKICCQFDFKRLPGGRFGCPWGVPPETIHPGNVQSRARMLLDQYRKKSKLFRTKVLLAPLGDDFRYCEYTEWDLQFKNYQQLFDYMNSQSKFKVKIQFGTLSDFFDALDKADETQRDKGQSMFPVLSGDFFTYADRDDHYWSGYFTSRPFYKRMDRIMESHLRAAEILYYFALRQAHKYKINKFLSSSLYTALTEARRNLGLFQHHDAITGTAKDWVVVDYGTRLFHSLMVLEKIIGNSAFLLILKDKLTYDSYSPDTFLEMDLKQKSQDSLPQKNIIRLSAEPRYLVVYNPLEQDRISLVSVYVSSPTVQVFSASGKPVEVQVSAVWDTANTISETAYEISFRAHIPPLGLKVYKILESASSNSHLADYVLYKNKVEDSGIFTIKNMINTEEGITLENSFVLLRFDQTGLMKQMMTKEDGKHHEVNVQFSWYGTTIKRDKSGAYLFLPDGNAKPYVYTTPPFVRVTHGRIYSEVTCFFDHVTHRVRLYHIQGIEGQSVEVSNIVDIRKVYNREIAMKISSDIKSQNRFYTDLNGYQIQPRMTLSKLPLQANVYPMTTMAYIQDAKHRLTLLSAQSLGVSSLNSGQIEVIMDRRLMQDDNRGLEQGIQDNKITANLFRILLEKRSAVNTEEEKKSVSYPSLLSHITSSLMNHPVIPMANKFSSPTLELQGEFSPLQSSLPCDIHLVNLRTIQSKVGNGHSNEAALILHRKGFDCRFSSKGTGLFCSTTQGKILVQKLLNKFIVESLTPSSLSLMHSPPGTQNISEINLSPMEISTFRIQLR

SEQ ID NO: 2 - protein, 1150 aa, Homo sapiens
MKLKKQVTVCGAAIFCVAVFSLYLMLDRVQHDPTRHQNGGNFPRSQISVLQNRIEQLEQLLEENHEIISHIKDSVLELTANAEGPPAMLPYYTVNGSWVVPPEPRPSFFSISPQDCQFALGGRGQKPELQMLTVSEELPFDNVDGGVWRQGFDISYDPHDWDAEDLQVFVVPHSHNDPGWIKTFDKYYTEQTQHILNSMVSKLQEDPRRRFLWAEVSFFAKWWDNINVQKRAAVRRLVGNGQLEIATGGWVMPDEANSHYFALIDQLIEGHQWLERNLGATPRSGWAVDPFGYSSTMPYLLRRANLTSMLIQRVHYAIKKHFAATHSLEFMWRQTWDSDSSTDIFCHMMPFYSYDVPHTCGPDPKICCQFDFKRLPGGRINCPWKVPPRAITEANVAERAALLLDQYRKKSQLFRSNVLLVPLGDDFRYDKPQEWDAQFFNYQRLFDFFNSRPNLHVQAQFGTLSDYFDALYKRTGVEPGARPPGFPVLSGDFFSYADREDHYWTGYYTSRPFYKSLDRVLEAHLRGAEVLYSLAAAHARRSGLAGRYPLSDFTLLTEARRTLGLFQHHDAITGTAKEAVVVDYGVRLLRSLVNLKQVIIHAAHYLVLGDKETYHFDPEAPFLQVDDTRLSHDALPERTVIQLDSSPRFVVLFNPLEQERFSMVSLLVNSPRVRVLSEEGQPLAVQISAHWSSATEAVPDVYQVSVPVRLPALGLGVLQLQLGLDGHRTLPSSVRIYLHGRQLSVSRHEAFPLRVIDSGTSDFALSNRYMQVWFSGLTGLLKSIRRVDEEHEQQVDMQVLVYGTRTSKDKSGAYLFLPDGEAKPYVPKEPPVLRVTEGPFFSEVVAYYEHIHQAVRLYNLPGVEGLSLDISSLVDIRDYVNKELALHIHTDIDSQGIFFTDLNGFQVQPRRYLKKLPLQANFYPMPVMAYIQDAQKRLTLHTAQALGVSSLKDGQLEVILDRRLMQDDNRGLGQGLKDNKRTCNRFRLLLERRTVGSEVQDSHSTSYPSLLSHLTSMYLNAPALALPVARMQLPGPGLRSFHPLASSLPCDFHLLNLRTLQAEEDTLPSAETALILHRKGFDCGLEAKNLGFNCTTSQGKVALGSLFHGLDVVFLQPTSLTLLYPLASPSNSTDVYLEPMEIATFRLRLG

SEQ ID NO: 3 - protein, 1150 aa, Mus musculus
MKLSRQFTVFGSAIFCVVIFSLYLMLDRGHLDYPRGPRQEGSFPQGQLSILQEKIDHLERLLAENNEIISNIRDSVINLSESVEDGPRGSPGNASQGSIHLHSPQLALQADPRDCLFASQSGSQPRDVQMLDVYDLIPFDNPDGGVWKQGFDIKYEADEWDHEPLQVFVVPHSHNDPGWLKTFNDYFRDKTQYIFNNMVLKLKEDSSRKFMWSEISYLAKWWDIIDIPKKEAVKSLLQNGQLEIVTGGWVMPDEATPHYFALIDQLIEGHQWLEKNLGVKPRSGWAIDPFGHSPTMAYLLKRAGFSHMLIQRVHYAIKKHFSLHKTLEFFWRQNWDLGSATDILCHMMPFYSYDIPHTCGPDPKICCQFDFKRLPGGRYGCPWGVPPEAISPGNVQSRAQMLLDQYRKKSKLFRTKVLLAPLGDDFRFSEYTEWDLQCRNYEQLFSYMNSQPHLKVKIQFGTLSDYFDALEKAVAAEKKSSQSVFPALSGDFFTYADRDDHYWSGYFTSRPFYKRMDRIMESRIRAAEILYQLALKQAQKYKINKFLSSPHYTTLTEARRNLGLFQHHDAITGTAKDWVVVDYGTRLFQSLNSLEKIIGDSAFLLILKDKKLYQSDPSKAFLEMDTKQSSQDSLPQKIIIQLSAQEPRYLVVYNPFEQERHSVVSIRVNSATVKVLSDSGKPVEVQVSAVWNDMRTISQAAYEVSFLAHIPPLGLKVFKILESQSSSSHLADYVLYNNDGLAENGIFHVKNMVDAGDAITIENPFLAIWFDRSGLMEKVRRKEDSRQHELKVQFLWYGTTNKRDKSGAYLFLPDGQGQPYVSLRPPFVRVTRGRIYSDVTCFLEHVTHKVRLYNIQGIEGQSMEVSNIVNIRNVHNREIVMRISSKINNQNRYYTDLNGYQIQPRRTMSKLPLQANVYPMCTMAYIQDAEHRLTLLSAQSLGASSMASGQIEVFMDRRLMQDDNRGLGQGVHDNKITANLFRILLEKRSAVNMEEEKKSPVSYPSLLSHMTSSFLNHPFLPMVLSGQLPSPAFELLSEFPLLQSSLPCDIHLVNLRTIQSKMGKGYSDEAALILHRKGFDCQFSSRGIGLPCSTTQGKMSVLKLFNKFAVESLVPSSLSLMHSPPDAQNMSEVSLSPMEISTFRIRLRWT

SEQ ID NO: 4 - protein, 1152 aa, Mus musculus
MKLKKQVTVCGAAIFCVAVFSLYLMLDRVQHDPARHQNGGNFPRSQISVLQNRIEQLEQLLEENHDIISRIKDSVLELTANAEGPPALLPYHTANGSWAVLPEPRPSFFSVSPQDCQFALGGRGQKPELQMLTVSEDLPFDNVEGGVWRQGFDISYSPNDWDTEDLQVFVVPHSHNDPGWIKTFDKYYTEQTQHILNSMVSKLQEDPRRRFLWAEVSFFAKWWDNISAQKRAAVRRLVGNGQLEIATGGWVMPDEANSHYFALVDQLIEGHQWLERNLGATPRSGWAVDPFGHSSTMPYLLRRANLTSMLIQRVHYAIKKHFAATHSLEFMWRQMWDSDSSTDIFCHMMPFYSYDVPHTCGPDPKICCQFDFKRLPGGRINCPWKVPPRAITEANVADRAALLLDQYRKKSRLFRSNVLLVPLGDDFRYDKPQEWDAQFFNYQRLFDFLNSKPEFHVQAQFGTLSEYFDALYKRTGVEPGARPPGFPVLSGDFFSYADREDHYWTGYYTSRPFYKSLDRVLEAHLRGAEILYSLALAHARRSGLAGQYPLSDFALLTEARRTLGLFQHHDAITGTAKEAVVVDYGVRLLRSLVSLKQVIINAAHYLVLGDQETYSFDPGTPFLQMDDSRVSHDALPERTVIRLDSSPRFVVVFNPLEQERLSVVSLLVNSPRVRVLSEEGQPLSVQISVHWSSATDMVPDVYQVSVPVRLPGLGLGVLQLQPDLDGPYTLQSSVRVYLNGVKLSVSRQSAFPVRVVDSGASDFAISNRYMQVWFSGLTGLLKSIRRVDEEQEQQMELEFLVYGTRTSKDKSGAYLFLPDSEAKPYVPKKPPVLRVTEGPFFSEVAVYYEHFHQVIRLYNLPGVEGLSLDMSFQVDIRDYVNKELALRIHTDIDSQGTFFTDLNGFQIQPRQYLKKLPLQANFYPMPVMAYIQDSQRRLTLHTAQALGVSSLGNGQLEVILDRRLMQDDNRGLGQGLKDNKITCNRFRLLLERRTTMSPEVHQEQERSTSYPSLLSHLTSMYLSTPPLVLPVAKRQGTSPALRSFHPLASPLPCDFHLLNLRMLPAEDTLPATDSALILHRKGFDCGLEAKNLGFNCTTSQGKLALGSLFHGLDVTFLQPTSLTLLYPLASPSNSTDISLEPMEISTFRLRLG

SEQ ID NO: 5 - protein, 1148 aa, Rattus norvegicus
MKLSRQFTVFGSAIFCVVIFSLYLMLDRGHLDYPRGPRQEGSFPQGQLSILQEKIDHLERLLAENNEIISNIRDSVINLSESVEDGPRGPAGNASQGSAHLHSAQLALQADPKDCLFASQSGNQHRDVQMLDVYDLIPFDNPDGGVWKQGFDIKYEADEWDREPLQVFVVPHSHNDPGWLKTFNDYFRDKTQYIFNNMVLKLKEDSSRKFIWSEISYLAKWWDIIDNPKKEAVKSLLQNGQLEIVTGGWVMADEATTHYFALIDQLIEGHQWLEKNLGVKPRSGWAIDPFGHSPTMTYLLKRAGFSHMLIQRVHYSVKKHFSLQKTLEFFWRQNWDLGSTTDILCHMMPFYSYDIPHTCGPDPKICCQFDFKRLPGGRYGCPWGVPPEAISPGNVQSRAQMLLDQYRKKSKLFRTKVLLAPLGDDFRFSEYTEWDLQYRNYEQLFSYMNSQPHLKVKIQFGTLSDYFDALEKSVAAEKKGGQSVFPALSGDFFTYADRDDHYWSGYFTSRPFYKRMDRIMESRLRTAEILYHLALKQAQKYKINKFLSSPHYTTLTEARRNLGLFQHHDAITGTAKDWVVVDYGTRLFQSLNSLEKIIGDSAFLLILKDKKLYQSDPSKAFLEMDTKQSSQDSLPKKNIIQLSAQEPRYLVVYNPFEQERHSVVSVRVNSATVKVLSDLGKAVEVQVSAVWKDMRTTSQAAYEVAFLAHLPPLGLKVYKILESQSSSSHLADYFLYNNDGQAESGIFHMKNMVDSGDAITIENSFLTLGFDRSGLMEKVRRKEDNKQQELKVQFLWYGTTNKRDKSGAYLFLPDGQGQPYVSLRTPFVRVTRGRIYSDVTCFLEHVTHKVRLYHIQGIEGQSMEVSNIVDIRSVHNREIVMRISSKINNQNRYYTDLNGYQIQPRRTMAKLPLQANVYPMSTMAYIQDAAHRLTLLSAQSLGASSMASGQIEVFMDRRLMQDDNRGLGQGVHDNKITANLFRILLEKRNGMNMEEDKKSPVSYPSLLSHMTSAFLNHPFLPMVLSGQLPSPAIELLSEFRLLQSSLPCDIHLVNLRTIQSKVGKGYSDEAALILHRKVFDCQLSSRAMGLPCSTTQGKMSIPKLFNNFAVESFIPSSLSLMHSPPDAQNTSEVSLSPMEISTSRIRLR

SEQ ID NO: 6 - protein, 1164 aa, Rattus norvegicus
MKLKKQVTVCGAAIFCVAVFSLYLMLDRVQHDPARHQNGGNFPRSQISVLQNRIEQLEQLLEENHEIISHIKDSVLELTANAEGPPALLPYHTANGSWAVLPEPRPSFFSVSPEDCQFALGGRGQKPELQMLTVSEDLPFDNVEGGVWRQGFDISYSPNDWDAEDLQVFVVPHSHNDPGWIKTFDKYYTEQTQHILNSMVSKLQEDPRRRFLWAEVSFFAKWWDNISAQKRAAVRRLVGNGQLEIATGGWVMPDEANSHYFALVDQLIEGHQWLERNLGATPRSGWAVDPFGHSSTMPYLLRRANLTSMLIQRVHYAIKKHFAATHSLEFMWRQTWDSDSSTDIFCHMMPFYSYDVPHTCGPDPKICCQFDFKRLPGGRINCPWKVPPRAITEANVADRAALLLDQYRKKSRLFRSSVLLVPLGDDFRYDKPQEWDAQFFNYQRLFDFLNSKPEFHVQAQFGTLSEYFDALYKRTGVEPGARPPGFPVLSGDFFSYADREDHYWTGYYTSRPFYKSLDRVLETHLRGAEVLYSLALAHARRSGLTGQYPLSDYAVLTEARRTLGLFQHHDAITGTAKEAVVVDYGVRLLRSLVSLKQVIINAAHYLVLGDKETYSFDPRAPFLQMDDSRVSHDALPERTVIRLDSSPRFVVVFNPLEQERLSVVSLLVNSPRVRVLSEEGQPLSVQISVQWSSATNMVPDVYQVSVPVRLPALGLGVLQLQPDLDGPYTLQSSVHVYLNGVKLSVSRQTTFPLRVVDSGTSDFAISNRYMQVWFSGLTGLLKSVRRVDEEQEQQVDMKLFVYGTRTSKDKSGAYLFLPDNEAKPYVPKKPPVLRVTEGPFFSEVAAYYEHFHQVIRLYNLPGVEGLSLDVSFQVDIRDYVNKELALRIHTDIDSQGTFFTDLNGFQVQPRKYLKKLPLQANFYPMPVMAYIQDSQRRLTLHTAQALGVSSLGNGQLEVILDRRLMQDDNRGLGQGLKDNKITCNHFRLLLERRTLMSPEVQQERSTSYPSLLSHMTSMYLNTPPLVLPVAKRESTSPTLHSFHPLASPLPCDFHLLNLRMLPAEVSVPVRANPHHQAEDTLPAADAALILHRKGFDCGLEAKNLGFNCTTSQGKLALGSLFHGLDVLFLQPTSLTLLYPLASPSNSTDISLEPMEISTFRLRLG

SEQ ID NO: 7 - protein, 447 aa, Homo sapiens
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 8 - protein, 442 aa, Rattus norvegicus
MRFRIYKRKVLILTLVVAACGFVLWSSNGRQRKNDALAPPLLDSEPLRGAGHFAASVGIRRVSNDSAAPLVPAVPRPEVDNLTLRYRSLVYQLNFDQMLRNVDKDGTWSPGELVLVVQVHNRPEYLRLLIDSLRKAQGIREVLVIFSHDFWSAEINSLISSVDFCPVLQVFFPFSIQLYPSEFPGSDPRDCPRDLKKNAALKLGCINAEYPDSFGHYREAKFSQTKHHWWWKLHFVWERVKVLQDYTGLILFLEEDHYLAPDFYHVFKKMWKLKQQECPGCDVLSLGTYTTIRSFYGIADKVDVKTWKSTEHNMGLALTRDAYQKLIECTDTFCTYDDYNWDWTLQYLTLACLPKVWKVLVPQAPRIFHAGDCGMHHKKTCRPSTQSAQIESLLNNNKQYLFPETLVIGEKFPMAAISPPRKNGGWGDIRDHELCKSYRRLQ

SEQ ID NO: 9 - protein, 442 aa, Mus musculus
MRFRIYKRKVLILTLVVAACGFVLWSSNGRQRKSDALGPPLLDAEPVRGAGHLAVSVGIRRVSNESAAPLVPAVPRPEVDNLTLRYRSLVYQLNFDQMLRNVGNDGTWSPGELVLVVQVHNRPEYLRLLIDSLRKAQGIQEVLVIFSHDFWSAEINSLISRVDFCPVLQVFFPFSIQLYPNEFPGSDPRDCPRDLKKNAALKLGCINAEYPDSFGHYREAKFSQTKHHWWWKLHFVWERVKVLQDYTGLILFLEEDHYLAPDFYHVFKKMWKLKQQECPGCDVLSLGTYTTIRSFYGIADKVDVKTWKSTEHNMGLALTRDAYQKLIECTDTFCTYDDYNWDWTLQYLTLACLPKIWKVLVPQAPRIFHAGDCGMHHKKTCRPSTQSAQIESLLNSNKQYLFPETLVIGEKFPMAAISPPRKNGGWGDIRDHELCKSYRRLQ

SEQ ID NO: 10 - protein, 442 aa, Cricetulus griseus
MRFRIYKRKVLILTLVVAACGFVLWSSNGRQRKNDGLAPPLLDAEPPRGPGHLAVSVGIRRVSNDSAPPVVPAAPRPEVDNLTLRYRSLVYQLNFDQMLRNVRDDGTWRPGELVLVVQVHNRPEYLRLLLDSLRKAQGINEVLVIFSHDFWSAEINHMIASVNFCPVLQVFFPFSIQLYPSEFPGSDPRDCPRDLKKNAALKLGCINAEYPDSFGHYREAKFSQTKHHWWWKLHFVWERVRVLQDYTGLILFLEEDHYLAPDFYHVFKKMWKLKQQECPGCDVLSLGTYTASRSFYGIADKVDVKTWKSTEHNMGLALTRDAYQKLIECTDTFCTYDDYNWDWTLQYLTVSCLPKFWKVLVPQAPRIFHAGDCGMHHKKTCRPSTQSAQIESFLNNNQQYMFPETLVISEKFSMAAISPPRKNGGWGDIRDHELCKSYRRLQ

SEQ ID NO: 11 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 399 aa, Rattus norvegicus
MRFREPFLGGSAAMPGATLQRACRLLVAVCALHLGVTLVYYLSGRDLSRLPQLVGVSSSLQGGTNGAAASKQPSGELRPRGARPPPPLGVSPKPRPGSDSSPDAASGPGLKSNLTSVPMPTSTGLLTLPACPEESPLLVGPMVIDFNIPVDLELLAKKNPEIKMGGRYFPKDCISPHKVAIIIPFRNRQEHLKYWLYYLHPVLQRQQLDYGIYVINQAGDTMFNRAKLLNVGFQEALKDYDYNCFVFSDVDLIPMDDHNAYRCFSQPRHISVAMDKFGFSLPYVQYFGGVSALSKQQFLTINGFPNNYWGWGGEDDDIFNRLVHKGMSISRPNAVVGRCRMIRHSRDKKNEPNPQRFDRIAHTKETMRLDGLNSLTYQVLDIQRYPLYTKITVDIGTPR

SEQ ID NO: 13 - protein, 399 aa, Mus musculus
MRFREQFLGGSAAMPGATLQRACRLLVAVCALHLGVTLVYYLSGRDLSRLPQLVGVSSTLQGGTNGAAASKQPPGEQRPRGARPPPPLGVSPKPRPGLDSSPGAASGPGLKSNLSSLPVPTTTGLLSLPACPEESPLLVGPMLIDFNIAVDLELLAKKNPEIKTGGRYSPKDCVSPHKVAIIIPFRNRQEHLKYWLYYLHPILQRQQLDYGIYVINQAGDTMFNRAKLLNIGFQEALKDYDYNCFVFSDVDLIPMDDRNAYRCFSQPRHISVAMDKFGFSLPYVQYFGGVSALSKQQFLAINGFPNNYWGWGGEDDDIFNRLVHKGMSISRPNAVVGRCRMIRHSRDKKNEPNPQRFDRIAHTKETMRFDGLNSLTYKVLDVQRYPLYTQITVDIGTPR

SEQ ID NO: 14 - protein, 393 aa, Cricetulus griseus
MRFLRPVLGGSAAMPGATLQRACRLLVAVCALHLGVTLVYYLSGRDLSRLPQLVGVSSTLRSGTIGATANKQPPGARPPPPVGVSSKPRPGPDSSPGTAFDPGLKSNWTSVLVPPTTALLTLPACPEESPLLVGPMVIDFNIAVDLELLAKKNPEIKMGGRYSPKDCISPHKVAIIIPFRNRQEHLKYWLYYLHPVLQRQQLDYGIYVINQAGDTMFNRAKLLNIGFQEALKDHDYNCFVFSDVDLIPMDDHNAYRCFSQPRHISVAMDKFGFSLPYVQYFGGVSALSKQQFLAINGFPNNYWGWGGEDDDIFNRIVHKGMSISRPNAVVGRCRMIRHSRDKKNEPNPQRFDRIAHTKETMRFDGLNSLTYQVLNVERYPLYTKITVDIGTPR

SEQ ID NO: 15 - protein, 403 aa, Rattus norvegicus
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDPSVYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYDPLLFEKNMVKHLNEGTDEDIYLFGKATLSGFRNIRC

SEQ ID NO: 16 - protein, 525 aa, Rattus norvegicus
MKPHLKQWRQRMLFAIFVWGLLFLAIFIYFTNSNPAAPMPSSFSFLESRGLLPVQGKQRVIMGALQEPSLPRSLEPSKVLMDGHSASPFNSWPGDPQKGDQAQDGFDNGDEFFTSQVGRKSQSAFYPEEDNYFFVAGQPGLYHHRQGALGLPSPGESSWQSGPGQPKQEKLRHPRRGSLPEEAYDSDMLSTSMSRAFLYRLWKGTVSSKMLNPRLQKAMRYYMSFNKHGVRFSRRGRREARRTGPELLCEMRKRVRVRTLDGKEAPFSGLGWRPLVPGVPLSQLHPRGLRSCAVVMSAGAILNSSLGEEIDSHDAVLRFNSAPTRGYEKDVGNKTTVRIINSQILANPSHHFIDSSLYKDVILVAWDPAPYSANLNLWYKKPDYNLFTPYIQHRLKYPTQPFYILHPKFIWQLWDIIQENTREKIQPNPPSSGFIGILVMMSMCQEVHVYEYIPSVRQTELCHYHELYYDAACTLGAYHPLLYEKLLVQRLNTGTQADLHHKGKVVLPGFQTLRCPVTRPNNTNT

SEQ ID NO: 17 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 18 - protein, 529 aa, Homo sapiens
MKPHLKQWRQRMLFGIFAWGLLFLLIFIYFTDSNPAEPVPSSLSFLETRRLLPVQGKQRAIMGAAHEPSPPGGLDARQALPRAHPAGSFHAGPGDLQKWAQSQDGFEHKEFFSSQVGRKSQSAFYPEDDDYFFAAGQPGWHSHTQGTLGFPSPGEPGPREGAFPAAQVQRRRVKKRHRRQRRSHVLEEGDDGDRLYSSMSRAFLYRLWKGNVSSKMLNPRLQKAMKDYLTANKHGVRFRGKREAGLSRAQLLCQLRSRARVRTLDGTEAPFSALGWRRLVPAVPLSQLHPRGLRSCAVVMSAGAILNSSLGEEIDSHDAVLRFNSAPTRGYEKDVGNKTTIRIINSQILTNPSHHFIDSSLYKDVILVAWDPAPYSANLNLWYKKPDYNLFTPYIQHRQRNPNQPFYILHPKFIWQLWDIIQENTKEKIQPNPPSSGFIGILIMMSMCREVHVYEYIPSVRQTELCHYHELYYDAACTLGAYHPLLYEKLLVQRLNMGTQGDLHRKGKVVLPGFQAVHCPAPSPVIPHS

SEQ ID NO: 19 - protein, 403 aa, Mus musculus
MIHTNLKRKFSCFVLVFLLFAIICVWKKGSDYEALTLQAKVFQMPKSQEKVAVGPAPQAVFSNSKQDPKEGVQILSYPRVTAKVKPQPSLQVWDKDSTYSKLNPRLLKIWRNYLNMNKYKVSYKGPGPGVKFSVEALRCHLRDHVNVSMIEATDFPFNTTEWEGYLPKENFRTKAGPWHKCAVVSSAGSLKNSQLGREIDNHDAVLRFNGAPTDNFQQDVGTKTTIRLVNSQLVTTEKRFLKDSLYTEGILILWDPSVYHADIPQWYQKPDYNFFETYKSYRRLHPSQPFYILKPQMPWELWDIIQEISPDLIQPNPPSSGMLGIIIMMTLCDQVDIYEFLPSKRKTDVCYYHQKFFDSACTMGAYHPLLFEKNMVKHLNEGTDEDIYLFGKATLSGFRNNRC

SEQ ID NO: 20 - protein, 524 aa, Mus musculus
MKPHLKQWRQRMLFGIFVWGLLFLAIFIYFTNSNPAAPMPSSFSFLERRGLLPLQGKQRVIMGALQEPSLPRSLDASKVLLDSHPENPFHPWPGDPQKWDQAPNGFDNGDEFFTSQVGRKSQSAFYPEEDSYFFVADQPELYHHRQGALELPSPGETSWRSGPVQPKQKLLHPRRGSLPEEAYDSDMLSASMSRAFLYRLWKGAVSSKMLNPRLQKAMRYYMSFNKHGVRFRRRGRREATRTGPELLCEMRRRVRVRTLDGREAPFSGLGWRPLVPGVPLSQLHPRGLSSCAVVMSAGAILNSSLGEEIDSHDAVLRFNSAPTRGYEKDVGNKTTVRIINSQILANPSHHFIDSALYKDVILVAWDPAPYSANLNLWYKKPDYNLFTPYIQHRRKYPTQPFYILHPKFIWQLWDIIQENTREKIQPNPPSSGFIGILIMMSMCKEVHVYEYIPSVRQTELCHYHELYYDAACTLGAYHPLLYEKLLVQRLNTGTQADLHHKGKVVLPGFQTLRCPVTSPNNTHS

SEQ ID NO: 21 - protein, 402 aa, Cricetulus griseus
MIHTNLKKKFSYFILAFLLFALICVWKKGSYEALKLQAKEFQVTRSLEKLAMRSGSQSMSSSSKQDPKQDSQVLSHARVTAKVKPQPSYQVWDKNSSSKNLNPRLQKILKNYLNMNKYKVSYKGPGPGVKFSAEALRCRLRDRVNVSMIEATDFPFNTTEWAGYLPKENIRTKAGPWHRCAVVSSAGSLKSSQLGREIDNHDAVLRFNGAPVANFQQDVGTKTTIRLMNSQLITTEKQFLKDSLYSEGILIVWDPSLYHADIPSWYQKPDYNFFETYKSYRKLYPDQPFYILRPQMPWELWDIIQEIAPDRIQPNPPSSGMLGIMIMMTLCDQVDIYEFLPSRRKTDVCYYHQKFFDSACTMGAYHPLLFEKNMVKQLNEGTDEDIYIFGKATLSGFRTIHC

SEQ ID NO: 22 - protein, 523 aa, Cricetulus griseus
  misc_feature (307)..(307): Xaa can be any naturally occurring amino acid
MKPHLKQWRQRMLFGIFVWGLLFLAIFIYFTNSNPASPVPSSFSFVENRGLLPVQGKQRAIMGALQESSLPRSLEASKALPGSHPASPFHGGPGDPQKWDQTQDGFDNGEEFFLPQVGRKSQSAFYPEEDSYFFAAGQPGWHRHTQGALGLPSPGEPSRRAGPVQHKREKLHRARRSRVPEDAYDGDMLSASMSRAFLYRLWKGTVSSKMLNPRLQKAMRYYMSFNKHGVRFRGRREVRRTGPELLCELRRRVRVRTLDGKEPPFSALGWRPLVPGVPLSQLHPRGLRTCAVVMSAGAILNSSLGEXIDSHDAVLRFNSAPTRGYEKDVGNKTTVRIINSQILANPSHHFIDSSLYKDVILVAWDPAPYSANLNLWYKKPDYNLFTPYIQHRRRHPTQPFYILHPKFIWQLWDIIQENTREKIQPNPPSSGFIGILIMKSMCREVHVYEYIPSVRQTELCHYHELYYDAACTLGAYHPLLYEKLLVQRLNMGTQADLHHKGKVVLPGFRALRCPVTSPNNTYS

SEQ ID NO: 23 - protein, 350 aa, Homo sapiens
MKCSLRVWFLSVAFLLVFIMSLLFTYSHHSMATLPYLDSGALDGTHRVKLVPGYAGLQRLSKERLSGKSCACRRCMGDAGASDWFDSHFDGNISPVWTRENMDLPPDVQRWWMMLQPQFKSHNTNEVLEKLFQIVPGENPYRFRDPHQCRRCAVVGNSGNLRGSGYGQDVDGHNFIMRMNQAPTVGFEQDVGSRTTHHFMYPESAKNLPANVSFVLVPFKVLDLLWIASALSTGQIRFTYAPVKSFLRVDKEKVQIYNPAFFKYIHDRWTEHHGRYPSTGMLVLFFALHVCDEVNVYGFGADSRGNWHHYWENNRYAGEFRKTGVHDADFEAHIIDMLAKASKIEVYRGN

SEQ ID NO: 24 - protein, 333 aa, Homo sapiens
MVSKSRWKLLAMLALVLVVMVWYSISREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 25 - protein, 331 aa, Homo sapiens
MRGYLVAIFLSAVFLYYVLHCILWGTNVYWVAPVEMKRRNKIQPCLSKPAFASLLRFHQFHPFLCAADFRKIASLYGSDKFDLPYGMRTSAEYFRLALSKLQSCDLFDEFDNIPCKKCVVVGNGGVLKNKTLGEKIDSYDVIIRMNNGPVLGHEEEVGRRTTFRLFYPESVFSDPIHNDPNTTVILTAFKPHDLRWLLELLMGDKINTNGFWKKPALNLIYKPYQIRILDPFIIRTAAYELLHFPKVFPKNQKPKHPTTGIIAITLAFYICHEVHLAGFKYNFSDLKSPLHYYGNATMSLMNKNAYHNVTAEQLFLKDIIEKNLVINLTQD

(19) United States
(12) Patent Application Publication
(10) Pub. No.: US 2015/0210777 A1
(43) Pub. Date: Jul. 30, 2015

(54) GLYCOPROTEIN

(71) Applicant: Glykos Finland Oy, Helsinki (FI)

(72) Inventors: Tero Satomaa, Helsinki (FI); Juhani Saarinen, Helsinki (FI); Jari Natunen, Vantaa (FI); Anja Vilkman, Klaukkala (FI); Heidi Virtanen, Helsinki (FI); Jukka Hiltunen, Helsinki (FI)

(21) Appl. No.: 14/365,014

(22) PCT Filed: Dec. 13, 2012
(86) PCT No.: PCT/FI2012/051238

§ 371 Date: Jun. 12, 2014

Related U.S. Application Data
(60) Provisional application No. 61/569,887, filed on Dec. 13, 2011.
(60) Provisional application No. 61/569,895, filed on Dec. 13, 2011.

(30) Foreign Application Priority Data
Dec. 13, 2011 (FI) 20116263

(51) Int. Cl. C07K 16/42; C07K 16/10; C07K 16/24; C07K 16/22; C07K 16/28

(52) U.S. Cl. CPC C07K 16/4291; C07K 16/22; C07K 16/2863; C07K 16/241; C07K 16/2887; C07K 16/2893; C07K 16/2896; C07K 16/2866; C07K 16/1027; A61K 2039/505

(57) ABSTRACT

The invention relates to a pharmaceutical composition comprising a glycoprotein comprising the Fc domain of an antibody, or a fragment thereof, comprising an Asn (asparagine) residue and an oligosaccharide structure attached thereto, wherein said oligosaccharide structure has a structure according to formula I, wherein at least 10% of the oligosaccharide structures attached to glycoproteins in the composition consist of oligosaccharide structures according to formula I.

### FIELD OF THE INVENTION

The invention relates to a glycoprotein, a composition, a host cell and a method of producing the glycoprotein or composition.

### BACKGROUND OF THE INVENTION

Glycoproteins mediate many essential functions in humans and other mammals, including signalling, cell-to-cell communication and molecular recognition and association. Antibodies or immunoglobulins are glycoproteins that play a central role in the humoral immune response and that are used increasingly as therapeutics. Antigen-specific recognition by antibodies results in the formation of immune complexes that may activate multiple effector mechanisms.

There are five major classes of immunoglobulins (Igs): IgA, IgD, IgE, IgG and IgM. Several of these may further be divided into subclasses (isotypes), e.g. IgG1, IgG2, IgG3 and IgG4. Papain digestion of antibodies produces two identical antigen binding fragments called Fab fragments and a residual Fc fragment. In human IgG molecules, the Fc region is generated by papain cleavage N-terminal to Cys 226. The Fc region is central to the effector function of the antibodies and interaction with various molecules, such as Fcγ receptors (FcγRI, FcγRIIa, FcγRIIb, FcγRIIc, FcγRIIIa and FcγRIIIb), rheumatoid factor (RF), Protein G and A, complement factors (C3b, C1q) and lectin receptors (MBL, MR, DC-SIGN (Dendritic Cell-Specific Intercellular adhesion molecule-3-Grabbing Non-integrin)). The interaction of antibodies and antibody-antigen complexes with cells of the immune system mediates a variety of responses, including antibody-dependent cell-mediated cytotoxicity (ADCC) and complement dependent cytotoxicity (CDC). In order to be useful in therapy, an antibody, or a fragment thereof, should therefore have suitable effector functions.

The Fc domain sequence of IgG comprises a single site for N-linked glycosylation within its C_{H}2 domain at an asparagine residue 297 (Asn297) numbered according to the EU index (Kabat et al., Sequences of proteins of immunological interest, 5^{th} ed., US Department of Health and Human Services, NIH Publication No. 91-3242). Typically the oligosaccharide structures attached to the Fc domain comprise biantennary chains with varying galactosylation.

It is known that the oligosaccharide structure attached to the Fc domain influences the binding of IgG to Fc receptors and other molecules that interact with the antibody molecule, such as DC-SIGN (Raju 2008, Curr Opin Immunol 20, 471-478). Thus variations in the oligosaccharide structure (i.e. different glycoforms) of the Fc domain influence ADCC and CDC activity. Subsequently, modification of said oligosaccharide structure may affect the therapeutic activity of an antibody or a fragment thereof. The ability to produce glycoproteins and compositions comprising thereof that are enriched for particular oligosaccharide structures is highly desirable.

### PURPOSE OF THE INVENTION

The purpose of the present invention is to disclose novel glycoproteins comprising an Fc domain and an oligosaccharide structure attached thereto that have decreased cytotoxic potential due to reduced affinity to Fc receptors. Another purpose of the present invention is to disclose said glycoproteins that have improved anti-inflammatory activity due to improved affinity to specific antibody receptors such as DC-SIGN.

### SUMMARY

The pharmaceutical composition according to the present invention is characterized by what is presented in claim 1.

The pharmaceutical composition according to the present invention is characterized by what is presented in claim 11.

The pharmaceutical composition or the glycoprotein for use in therapy according to the present invention is characterized by what is presented in claim 16.

The host cell according to the present invention is characterized by what is presented in claim 18.

The method of treating autoimmune diseases, inflammatory disorders or any other disease where binding to an antibody target or increased anti-inflammatory activity with reduced cytotoxic activity is desired according to the present invention is characterized by what is presented in claim 22.

The method for producing the glycoprotein according to the present invention is characterized by what is presented in claim 23.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 shows MALDI-TOF mass spectrometric characterization of humanized IgG1 antibody glycoforms. N-glycans were liberated and analyzed as [M+Na]+ ions (m/z on the x-axis). A. Hybrid-type glycoform. B. Monoantennary glycoform;

FIG. 2 shows MALDI-TOF mass spectrometric characterization of humanized IgG1 antibody α2,6-sialylated hybrid-type glycoform. N-glycans were liberated and analyzed as [M+Na]+ ions (m/z on the x-axis);

FIG. 3 shows DC-SIGN binding results (relative affinity on the y-axis) of humanized IgG1 antibody glycoforms; and

FIG. 4 displays C1q binding results (relative affinity on the y-axis) of humanized IgG1 antibody glycoforms.

### DETAILED DESCRIPTION OF THE INVENTION

The present inventors have surprisingly found that a certain subset of oligosaccharide structures present in glycoproteins comprising an Fc domain or a fragment thereof mediate greatly reduced cytotoxicity and improved anti-inflammatory activity as compared to oligosaccharide structures typically present in said glycoproteins. This effect is due to e.g. reduced ADCC and CDC activity and improved binding to molecules such as DC-SIGN.

The present invention relates to a glycoprotein comprising the Fc domain of an antibody, or a fragment thereof, comprising an Asn (asparagine) residue and an oligosaccharide structure attached thereto, wherein said oligosaccharide structure has a structure according to formula I

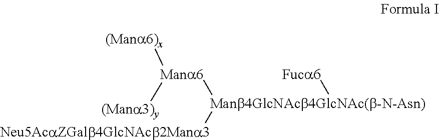

wherein
(β-N-Asn)=β-N linkage to Asn;

### Z=3 or 6;

x=0 or 1; and
y=0 or 1.

The glycoprotein of the invention comprises the Fc domain of an IgG molecule, or a fragment thereof, which comprises a site for N-linked glycosylation at an Asn residue.

In this context, the term “Fc domain” should be understood as meaning a C-terminal region of an antibody or an immunoglobulin heavy chain (“antibody” and “immunoglobulin” are used herein interchangeably). Although the boundaries of the Fc domain of an immunoglobulin heavy chain might vary, the human IgG heavy chain Fc domain is usually defined to stretch from an amino acid residue at position Cys226 to the carboxyl-terminus thereof. The Fc domain generally comprises two constant domains, CH2 and CH3. The “CH2 domain” of a human IgG Fc domain usually extends from about amino acid 231 to about amino acid 340. The “CH3 domain” of a human IgG Fc domain usually extends from about amino acid 341 to about amino acid residue 447 of a human IgG (i.e. comprises the residues C-terminal to a CH2 domain). The term “Fc domain” is also intended to include naturally occurring allelic variants of the “Fc domain” as well as variants having alterations which produce substitutions, additions, or deletions but which do not decrease substantially the ability of the Fc domain to bind effector molecules such as Fc receptors or mediate antibody dependent cellular cytotoxicity. For example, one or more amino acids can be deleted from the N-terminus or C-terminus of the Fc domain of an immunoglobulin without substantial loss of biological function. Such variants, or fragments, of an Fc domain can be selected according to general rules known in the art (See, e.g., Bowie, J. U. et al., Science 247:1306-10 (1990).

In one embodiment of the invention, the Asn residue corresponds to asparagine at position 297 (Asn297) of human IgG wherein the numbering corresponds to the EU index according to Kabat. In this context, the term “according to Kabat” should be understood as meaning the numbering as described in Kabat et al., Sequences of proteins of immunological interest, 5^{th} ed., US Department of Health and Human Services, NIH Publication No. 91-3242. A person skilled in the art can easily identify the amino acid residue corresponding to Asn297 by performing a sequence alignment. The amino acid residue corresponding to Asn297 will align with Asn297. While Asn297 is the N-glycosylation site typically found in murine and human IgG molecules, this site is not the only site that can be envisioned, nor does this site necessarily have to be maintained. Using known methods for mutagenesis, a skilled person can alter a DNA molecule encoding an Fc domain of the present invention so that the N-glycosylation site at Asn297 is deleted, and can further alter the DNA molecule so that one or more N-glycosylation sites are created at other positions within the Fc_domain. It is preferred that N-glycosylation sites are created within the CH2 region of the antibody molecule.

In one embodiment of the present invention, the Fc domain comprises two heavy chain sequences each comprising at least one Asn residue. In one embodiment of the present invention, one or two of the Fc domain Asn residues are N-glycosylated with oligosaccharide structure according to the invention. In a preferred embodiment of the present invention, two Fc domain Asn residues are N-glycosylated with oligosaccharide structures according to the invention.

In one embodiment of the present invention, the glycoprotein is capable of interacting with at least one molecule selected from the group consisting of FcγRI, FcγRIIa, FcγRIIb, FcγRIIc, FcγRIIIa, FcγRIIIb, rheumatoid factor, Protein G, protein A, C3b, C1q, MBL, MR, and DC-SIGN.

In one embodiment of the present invention, the glycoprotein exhibits reduced interaction with at least one molecule selected from the group consisting of FcγRI, FcγRIIa, FcγRIIc, FcγRIIIa, FcγRIIIb, C1q and C3b. In this context, the term “reduced interaction” should be understood as meaning reduced interaction as compared with a glycoprotein comprising a normal oligosaccharide structure attached thereto.

In this context, the term “normal oligosaccharide structure” should be understood as meaning an N-glycan structure commonly found attached to an Fc domain shown in the following formula:

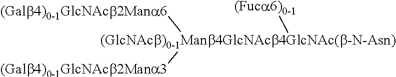

wherein
(β-N-Asn)=β-N linkage to Asn; and the notation 0-1 in e.g. (Galβ4)₀₋₁ should be understood as meaning either absent (0) or present (1); in other words, the notation (Galβ4)₀ means that the Gal residue is not present, and the notation (Galβ4)₁ means that one Gal residue is present. In this context, the term “normal glycoform” should be understood as meaning a glycoprotein comprising a normal oligosaccharide structure. Said normal oligosaccharide structure is present in the majority of antibodies and other glycoproteins comprising an Fc domain produced in mammalian cells.

In this context, the term “hybrid-type oligosaccharide structure” should be understood as meaning an N-glycan structure shown in the formula below:

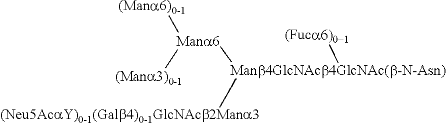

wherein Y=3 or 6; (β-N-Asn)=β-N linkage to Asn; and the notation 0-1 in e.g. (Galβ4)₀₋₁ should be understood as meaning either absent or present; in other words, the notation (Galβ4)₀ means that the Gal residue is not present, and the notation (Galβ4)₁ means that one Gal residue is present; when Neu5Ac is present also Gal is present; and at least one of the optional Manα6 and Manα3 groups is present. In this context, the term “hybrid-type glycoform” should be understood as meaning a glycoprotein comprising a hybrid-type oligosaccharide structure. Specifically, the term “sialylated hybrid-type oligosaccharide structure” should be understood as meaning the hybrid-type oligosaccharide structure wherein Neu5Ac is present. The term “sialylated hybrid-type glycoform” should be understood as meaning a glycoprotein comprising a sialylated hybrid-type oligosaccharide structure.

In this context, the term “monoantennary oligosaccharide structure” should be understood as meaning an N-glycan structure shown in the formula below:

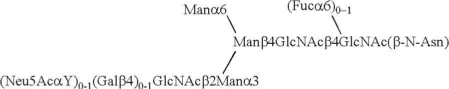

wherein Y=3 or 6; (β-N-Asn)=β-N linkage to Asn; and the notation 0-1 in e.g. (Galβ4)₀₋₁ should be understood as meaning either absent or present; in other words, the notation (Galβ4)₀ means that the Gal residue is not present, and the notation (Galβ4)₁ means that one Gal residue is present; when Neu5Ac is present also Gal is present. In this context, the term “monoantennary glycoform” should be understood as meaning a glycoprotein comprising a monoantennary oligosaccharide structure. Specifically, the term “sialylated monoantennary oligosaccharide structure” should be understood as meaning the monoantennary structure wherein Neu5Ac is present, and the term “sialylated monoantennary glycoform” should be understood as meaning a glycoprotein comprising a sialylated monoantennary oligosaccharide structure.

In one embodiment of the present invention, the glycoprotein exhibits improved interaction with DC-SIGN. In this context, the term “improved interaction” should be understood as meaning improved interaction as compared with a glycoprotein comprising normal oligosaccharide structure. This embodiment has improved anti-inflammatory activity. In one embodiment a glycoprotein of the invention exhibits improved interaction with DC-SIGN, as compared to the glycoprotein comprising normal oligosaccharide structure. In some embodiments, the interaction of the glycoprotein with DC-SIGN is improved by about 1.20 fold to about 100 fold, or about 1.5 fold to about 50 fold, or about 2 fold to about 25 fold, as compared to the glycoprotein comprising normal oligosaccharide structure, where interaction is determined e.g. as disclosed in the Examples herein. In other embodiments, the interaction of the glycoprotein with DC-SIGN is improved by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, as compared to the glycoprotein comprising normal oligosaccharide structure, where interaction is determined as disclosed in the Examples herein.

In one embodiment of the present invention, the glycoprotein exhibits reduced ADCC. In this context, the term “reduced ADCC” should be understood as meaning reduced ADCC as compared with a glycoprotein comprising normal oligosaccharide structure. This embodiment has reduced cytotoxic activity. ADCC may be measured e.g. using the TNF-α production assay described in Example 3. In certain embodiments, a glycoprotein of the invention has reduced ADCC or CDC activity, as compared to the glycoprotein comprising normal oligosaccharide structure. In some embodiments, ADCC or CDC activity is reduced by about 1.20 fold to about 100 fold, or about 1.5 fold to about 50 fold, or about 2 fold to about 25 fold, as compared to the glycoprotein comprising normal oligosaccharide structure. In other embodiments, the ADCC or CDC activity of a glycoprotein is reduced by at least about 1.10 fold, 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, or at least about 25 fold, as compared to the glycoprotein comprising normal oligosaccharide structure.

In one embodiment a glycoprotein of the invention exhibits decreased interaction with at least one effector molecule, as compared to the glycoprotein comprising normal oligosaccharide structure. In this context, the term “effector molecule” should be understood as meaning a molecule selected from the group consisting of FcγRI, FcγRIIa, FcγRIIc, FcγRIIIa, FcγRIIIb, C1q and C3b, as compared to the glycoprotein comprising normal oligosaccharide structure. In some embodiments, the interaction of the glycoprotein with an effector molecule is decreased by about 1.20 fold to about 100 fold, or about 1.5 fold to about 50 fold, or about 2 fold to about 25 fold, as compared to the glycoprotein comprising normal oligosaccharide structure, where interaction is determined e.g. as disclosed in the Examples herein. In other embodiments, the interaction of the glycoprotein with an effector molecule is decreased by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, where effector molecule interaction is determined as disclosed in the Examples herein. In one embodiment, the effector molecule that the glycoprotein has decreased interaction with is Fc□RIIIa. In one embodiment, the effector molecule that the glycoprotein has decreased interaction with is C1q.

In this context, the term “oligosaccharide structure” should be understood as meaning glycan structure or portions thereof, which comprises sugar residues. Such sugar residues may comprise e.g. mannose, N-acetylglucosamine, glucose, galactose, sialic acid or fucose linked to each other through glycosidic bonds in a particular configuration.

In one embodiment of the present invention, the term “oligosaccharide structure” should be understood as meaning an N-glycan.

A person skilled in the art will appreciate that glycoproteins are typically produced in vivo and in vitro as a plurality of variants comprising a mixture of specific oligosaccharide structures attached thereto. In other words, glycoproteins are typically present as different glycoforms.

In this context, the term “glycoform” should be understood as meaning a glycoprotein of the invention comprising specific oligosaccharide structures sharing a common structural feature.

As known in the art (see e.g. “Essentials of Glycobiology”, 2^{nd} edition, Ed. Varki, Cummings, Esko, Freeze, Stanley, Bertozzi, Hart & Etzler; Cold Spring Harbor Laboratory Press, 2009) and used herein, the term “glycan” should be understood to refer to homo- or heteropolymers of sugar residues, which may be linear or branched. “N-glycan”, a term also well known in the art, refers to a glycan conjugated by a β-N-linkage (nitrogen linkage through a β-glycosidic bond) to an asparagine (Asn) residue of a protein. Carbohydrate nomenclature in this context is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293).

In this context, the abbreviation “Man” should be understood as meaning D-mannose; “GlcNAc” refers to N-acetyl-D-glucosamine (2-acetamido-2-deoxy-D-glucose); “Fuc” refers to L-fucose; “Gal” refers to D-galactose; terms “Neu5Ac”, “NeuNAc” and “sialic acid” refer to N-acetylneuraminic acid; all monosaccharide residues are in pyranose form; all monosaccharides are D-sugars except for L-fucose; “Hex” refers to a hexose sugar; “HexNAc” refers to an N-acetylhexosamine sugar; and “dHex” refers to a deoxyhexose sugar. In one embodiment of the present invention, “sialic acid” may also refer to other sialic acids in addition to N-acetylneuraminic acid, such as N-glycolylneuraminic acid (Neu5Gc). The notation of the oligosaccharide structure and the glycosidic bonds between the sugar residues comprised therein follows that commonly used in the art, e.g. “Manα2Man” should be understood as meaning two mannose residues linked by a covalent linkage between the first carbon atom of the first mannose residue to the second carbon atom of the second mannose residue linked by an oxygen atom in the alpha configuration. Furthermore, in this context, the notation of the oligosaccharide structure “Neu5AcαYGalβ” wherein Y=3 or 6 should be understood as meaning a structure comprising a N-acetylneuraminic acid residue linked to a galactose residue by a covalent linkage between the second carbon atom of the N-acetylneuraminic acid residue to either the third or the sixth carbon atom of the galactose residue linked by an oxygen atom in the alpha configuration.

In this context, the notation “Neu5Acα3Galβ4GlcNAcβ2Manα3(Manα6)Manβ4GlcNAcβ4(Fucα6) GlcNAc” should be understood as referring to an oligosaccharide structure according to formula I wherein x=0 and y=0. Brackets and square brackets in the context of this type of notation indicate branches in the oligosaccharide structure.

In one embodiment of the present invention, the glycoprotein comprises the oligosaccharide structure having the structure according to formula I wherein x=1 and y=1. This embodiment has the effect that the presence of three Man residues leads to effective fucosylation, galactosylation and sialylation of the oligosaccharide structure when the glycoprotein of the invention is produced in mammalian cell culture.

In one embodiment of the present invention, the glycoprotein comprises the oligosaccharide structure having the structure according to formula I wherein x=0 and y=0.

The present invention further relates to a glycoprotein comprising the Fc domain of an antibody, or a fragment thereof, comprising an Asn residue and an oligosaccharide structure attached thereto, wherein the oligosaccharide structure has a structure according to formula II

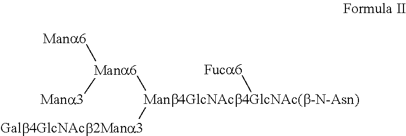

In other words, said oligosaccharide structure has the structure according to formula I wherein x=1 and y=1 without the presence of Neu5Ac. This embodiment has the effect that the presence of three Man residues leads to effective fucosylation and galactosylation of the oligosaccharide structure when the glycoprotein of the invention is produced in mammalian cell culture.

The present invention further relates to a composition comprising a glycoprotein comprising the Fc domain of an antibody, or a fragment thereof, comprising an Asn residue and an oligosaccharide structure attached thereto, wherein the oligosaccharide structure attached to glycoprotein in the composition consist of oligosaccharide structures according to formula II.

In one embodiment of the invention, at least 66.7% (⅔) of the oligosaccharide structures attached to glycoprotein in the composition consist of oligosaccharide structures according to formula II.

In one embodiment of the invention, at least 80% of the oligosaccharide structures attached to glycoprotein in the composition consist of oligosaccharide structures according to formula II.

In one embodiment of the invention, at least 90%, or at least 95%, or at least 98%, or at least 99%, or at least 99.5%, or essentially all of the oligosaccharide structures attached to glycoprotein in the composition consist of oligosaccharide structures according to formula II.

In one embodiment of the present invention, the glycoprotein comprises an Fc domain which is a human Fc domain, or a fragment thereof.

In one embodiment of the present invention, the glycoprotein is a fusion protein comprising an Fc domain, or a fragment thereof. Said fusion protein may, in addition to the Fc domain, or a fragment thereof, comprise e.g. a receptor moiety having a different biological function. The fusion protein should also be understood as meaning antibody like molecules which combine the “binding domain” of a heterologous “adhesin” protein (e.g. a receptor, ligand or enzyme) with an Fc domain. Structurally, these immunoadhesins comprise a fusion of the adhesin amino acid sequence with the desired binding specificity which is other than the antigen recognition and binding site (antigen combining site) of an antibody (i.e. is “heterologous”) and an Fc domain sequence. Examples of immunoadhesins include, but are not limited to, etanercept (available e.g. under the trade mark ENBREL®), which is a soluble TNF receptor 2 protein fused to the Fc region of human IgG1, carcinoembryonic antigen-immunoglobulin Fc fusion protein and factor IX-Fc fusion protein.

In one embodiment of the present invention, the glycoprotein comprises a fusion protein comprising an Fc domain, or a fragment thereof.

In one embodiment of the invention, the glycoprotein is a human antibody. In this context, the term “human antibody”, as it is commonly used in the art, is to be understood as meaning antibodies having variable regions in which both the framework and complementary determining regions (CDRs) are derived from sequences of human origin.

In one embodiment of the invention, the glycoprotein comprises a human antibody.

In one embodiment of the invention, the glycoprotein is a humanized antibody. In this context, the term “humanized antibody”, as it is commonly used in the art, is to be understood as meaning antibodies wherein residues from a CDR of an antibody of human origin are replaced by residues from a CDR of a nonhuman species (such as mouse, rat or rabbit) having the desired specificity, affinity and capacity.

In one embodiment of the invention, the glycoprotein comprises a humanized antibody.

In one embodiment of the invention, the glycoprotein is a chimeric antibody comprising a human Fc domain. In this context, the term “chimeric antibody”, as it is commonly used in the art, is to be understood as meaning antibodies wherein residues in an antibody of human origin are replaced by residues from an antibody of a nonhuman species (such as mouse, rat or rabbit) having the desired specificity, affinity and capacity.

In one embodiment of the invention, the glycoprotein comprises a chimeric antibody comprising a human Fc domain.

In this context, the terms “antibody” and “immunoglobulin”, as commonly used in the art, should be understood as being used interchangeably.

In one embodiment of the invention, the glycoprotein is an IgG (immunoglobulin G) antibody.

In one embodiment of the invention, the glycoprotein comprises an IgG (immunoglobulin G) antibody.

In one embodiment of the invention, the glycoprotein is an IgG1, IgG2, IgG3 or IgG4 antibody.

In one embodiment of the invention, the glycoprotein comprises an IgG1, IgG2, IgG3 or IgG4 antibody.

In one embodiment of the present invention, the glycoprotein is a monoclonal antibody.

In one embodiment of the present invention, the glycoprotein is an antibody directed against human vascular endothelial growth factor (VEGF), epidermal growth factor receptor 1 (EGFR), tumor necrosis factor alpha (TNF-α), CD20, epidermal growth factor receptor (HER2/neu), CD52, CD33, CD11a, glycoprotein IIb/IIIa, CD25, IgE, IL-2 receptor, or respiratory syncytial virus (RSV). However, these antibody targets are provided as examples only, to which the invention is not limited; a skilled person will appreciate that the glycoprotein of the invention is not limited to any particular antibody or form thereof. In one embodiment of the present invention, the glycoprotein is the antibody bevacizumab (available e.g. under the trademark AVASTIN®), tositumomab (BEXXAR®), etanercept (ENBREL®), trastuzumab (HERCEPTIN®), Adalimumab (HUMI-RA®), alemtuzumab (CAMPATH®), gemtuzumab ozogamicin (MYLOTARG®), efalizumumab (RAPTIVE®), rituximab (RITUXAN®), infliximab (REMICADE®), abciximab (RE-OPRO®), baasiliximab (SIMULECT®), palivizumab (SYN-AGIS®), omalizumab (XOLAIR®), daclizumab (ZENAPAX®), cetuximab (ERBITUX®), panitumumab (VECTIBIX®) or ibritumomab tiuxetan (ZEVALIN®). However, these antibodies are provided as examples only, to which the invention is not limited; a skilled person will appreciate that the glycoprotein of the invention is not limited to any particular antibody or form thereof.

Monoclonal antibodies to the target of interest may be prepared using any technique which provides for the production of antibody molecules by continuous cell lines in culture. These include, but are not limited to, the hybridoma technique originally described by Kohler and Milstein, 1975, Nature 256:495-497, the human B-cell hybridoma technique (Kosbor et al., 1983, Immunology Today 4:72; Cote et al., 1983, Proc. Natl. Acad. Sci. U.S.A. 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce single chain antibodies having a desired specificity.

In one embodiment of the present invention, the glycoprotein further comprises a conjugated molecule selected from a group consisting of a detection-enabling molecule and a therapy-enabling molecule. Examples of detection-enabling molecules are molecules conveying affinity such as biotin or a His tag comprising at least five histidine (His) residues; molecules that have enzymatic activity such as horseradish peroxidase (HRP) or alkaline phosphatase (AP); various fluorescent molecules such as FITC, TRITC, and the Alexa and Cy dyes; gold; radioactive atoms or molecules comprising such; chemiluminescent or chromogenic molecules and the like, which molecules provide a signal for visualization or quantitation. A therapy-enabling molecule may be a molecule used for e.g. increasing valence, size, stability and/or prolonged circulation of antibodies and other therapeutic proteins, e.g. a polyethylene glycol (PEG) or poly(vinylpyrrolidone) (PVP) moiety, a radioactive atom or molecule comprising said atom to be used for radiotherapy, or e.g. a toxin or a prodrug activating enzyme.

The present invention also relates to a composition comprising the glycoprotein of the present invention.

In one embodiment of the invention, the composition further comprises a glycoprotein having a different oligosaccharide structure. In other words, the composition further comprises one or more glycoforms.

In one embodiment of the invention, at least 10% of the oligosaccharide structures attached to glycoprotein in the composition consist of oligosaccharide structures according to formula I.

In one embodiment of the invention, at least 50% of the oligosaccharide structures attached to glycoprotein in the composition consist of oligosaccharide structures according to formula I.

In one embodiment of the invention, at least 66.7% (⅔) of the oligosaccharide structures attached to glycoprotein in the composition consist of oligosaccharide structures according to formula I.

In one embodiment of the invention, at least 80% of the oligosaccharide structures attached to glycoprotein in the composition consist of oligosaccharide structures according to formula I.

In one embodiment of the invention, at least 90% of the oligosaccharide structures attached to glycoprotein in the composition consist of oligosaccharide structures according to formula I.

In one embodiment of the invention, at least 95% of the oligosaccharide structures attached to glycoprotein in the composition consist of oligosaccharide structures according to formula I.

In one embodiment of the present invention, the feature “at least 10% of the oligosaccharide structures attached to glycoprotein in the composition consist of oligosaccharide structures according to formula I” or any other feature indicating the percentage or the proportion of specific oligosaccharide structures should be understood as referring to a feature indicating that the indicated proportion, e.g. at least 10%, of all oligosaccharide structures attached to any glycoprotein in the composition, said any glycoprotein comprising a glycoprotein of the invention and optionally one or more other glycoproteins, consist of the specific oligosaccharide structures, e.g. those according to formula I. The percentage or proportion of oligosaccharide structures or portions thereof attached to glycoprotein or glycoproteins in the composition may be measured e.g. by releasing all oligosaccharide structures attached to any glycoprotein in the composition and determining the percentage or proportion of the specific oligosaccharide structures therein, as described e.g. in the Examples.

In one embodiment of the present invention, the feature “at least 10% of the oligosaccharide structures attached to glycoprotein in the composition consist of oligosaccharide structures according to formula I” or any other feature indicating the percentage or the proportion of specific oligosaccharide structures should be understood as referring to a feature indicating that the indicated proportion, e.g. at least 10%, of the Fc domain oligosaccharide structures attached to the Fc domains in the composition, said Fc domains comprised in a glycoprotein of the invention and optionally in one or more other glycoproteins, consist of the specific oligosaccharide structures, e.g. those according to formula I. The percentage or proportion of oligosaccharide structures or portions thereof attached to said Fc domain or Fc domains in the composition may be measured e.g. by isolating the Fc domains or antibodies in the composition, releasing all oligosaccharide structures attached to the Fc domains and determining the percentage or proportion of the specific oligosaccharide structures therein, as described e.g. in the Examples.

In one embodiment of the invention, the composition is a pharmaceutical composition comprising a glycoprotein comprising the Fc domain of an antibody, or a fragment thereof, comprising an Asn residue and an oligosaccharide structure attached thereto, characterised in that the oligosaccharide structure has a structure according to formula I wherein

(β-N-Asn)=β-N linkage to Asn;

Z=3 or 6;

x=0 or 1; and y=0 or 1;

wherein at least 10% of the oligosaccharide structures attached to glycoproteins in the composition consist of oligosaccharide structures according to formula I.

In one embodiment of the present invention, at least 50%, or at least 66.7%, or at least 80%, or at least 90% of the oligosaccharide structures attached to glycoproteins in the composition consist of oligosaccharide structures according to formula I.

In one embodiment of the invention, at least 50%, or at least 66.7%, or at least 80%, or at least 90% of the oligosaccharide structures attached to glycoproteins in the composition consist of oligosaccharide structures according to formula I.

In one embodiment of the present invention, the composition of the invention further comprises a glycoprotein comprising the Fc domain of an antibody, or a fragment thereof, comprising an Asn residue and an oligosaccharide structure attached thereto, wherein the oligosaccharide structure has a structure according to formula III

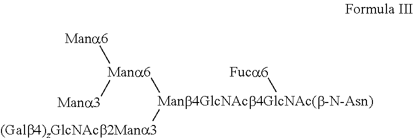

wherein
(β-N-Asn)=β-N linkage to Asn;

z=0 or 1; and

wherein at least 10% of the oligosaccharide structures attached to glycoprotein in the composition consist of oligosaccharide structures according to formula III.

In one embodiment of the present invention, at least 50%, 60%, 70%, 80%, 90%, 95%, 98%, 99%, 99.5% or essentially all of the oligosaccharide structures attached to glycoprotein in the composition consist of oligosaccharide structures according to formula I and of oligosaccharide structures according to formula III.

In one embodiment of the invention, at least 95% of the oligosaccharide structures attached to glycoprotein in the composition comprise α1,6-linked fucose (Fuc) residue. Said fucose residue, as shown in formula I, is attached to the GlcNAc residue present in the core Manβ4GlcNAcβ4GlcNAc structure that is linked by a β-N linkage to Asn. In other words, at least 95% of the oligosaccharide structures attached to glycoproteins in the composition are core fucosylated.

In this context, the term “core fucosylated” should be understood as meaning an oligosaccharide structure wherein a Fuc residue, as shown in formula I, is attached to the core GlcNAc residue present in the core Manβ4GlcNAcβ4GlcNAc structure that is linked by a β-N linkage to Asn.

In one embodiment of the invention, at least 98% of the oligosaccharide structures attached to glycoprotein in the composition comprise the Fuc residue.

In one embodiment of the invention, at least 99% of the oligosaccharide structures attached to glycoprotein in the composition comprise the Fuc residue.

In one embodiment of the invention, at least 99.5% of the oligosaccharide structures attached to glycoprotein in the composition comprise the Fuc residue.

In one embodiment of the invention, essentially all (100%) oligosaccharide structures attached to glycoprotein in the composition comprise the α1,6-linked fucose residue.

In one embodiment of the present invention, the composition is a pharmaceutical composition.

In this context, the term “pharmaceutical composition” should be understood as a composition for administration to a patient, preferably a human patient.

In one embodiment of the present invention, the pharmaceutical composition comprises a composition for e.g. oral, parenteral, transdermal, intraluminal, intraarterial, intrathecal and/or intranasal administration or for direct injection into tissue. Administration of the pharmaceutical composition may be effected in different ways, e.g. by intravenous, intraperitoneal, subcutaneous, intramuscular, topical or intradermal administration. The pharmaceutical composition of the present invention may further comprise a pharmaceutically acceptable carrier. Examples of suitable pharmaceutically acceptable carriers are well known in the art and include e.g. phosphate buffered saline solutions, water, oil/water emulsions, wetting agents, and liposomes. Compositions comprising such carriers may be formulated by methods well known in the art. Dosages and dosage regimens, as known in the art, may vary depending on a number of factors and may be determined depending on e.g. the patient's age, size, the nature of the glycoprotein, and the administration route. The pharmaceutical composition may further comprise other components such as vehicles, additives, preservatives, other pharmaceutical compositions administrated concurrently, and the like.

The present invention further relates to the glycoprotein or composition according to the invention for use in therapy.

In one embodiment of the present invention, the glycoprotein or composition is administered in a therapeutically effective amount to a human or animal.

The present invention further relates to the glycoprotein or composition according to the invention for use in the treatment of autoimmune diseases, inflammatory disorders or any other disease where binding to an antibody target or increased anti-inflammatory activity with reduced cytotoxic activity is desired.

In one embodiment of the present invention, the term “increased anti-inflammatory activity” should be understood as meaning improved interaction with DC-SIGN. In this context, the term “improved interaction” should be understood as meaning improved interaction as compared with a glycoprotein comprising normal oligosaccharide structure.

In one embodiment of the present invention, the term “reduced cytotoxic activity” should be understood as meaning reduced ADCC. In this context, the term “reduced ADCC” should be understood as meaning reduced ADCC as compared with a glycoprotein comprising normal oligosaccharide structure.

The present invention further relates to a host cell comprising a polynucleotide encoding the protein moiety of a glycoprotein according to the invention, wherein said host cell has reduced activity of mannosidase II compared to the parent cell.

“Activity of mannosidase II” should be understood as meaning correlation between a level of mannosidase II enzyme activity to hydrolyze Manα3 and Manα6 residues in the oligosaccharide structure according to Formula I attached to the glycoprotein of the invention and % portion of the Manα3 and Manα6 residues in the oligosaccharide structures according to formula I attached to glycoproteins in the composition of the invention. A host cell has “reduced or decreased activity of mannosidase II” when said cell produces higher % portion of the Manα3 and Manα6 residues in the oligosaccharide structures according to formula I attached to glycoproteins in the composition of the invention when cultured in similar or identical conditions compared to parent cell without manipulations to decrease mannosidase II activity.

In this context, the term “host cell” should be understood as meaning any cell suitable for producing the glycoprotein of the invention.

In this context, the term “protein moiety” should be understood as meaning the glycoprotein without the oligosaccharide structure attached.

In one embodiment of the present invention, the host cell produces the glycoprotein of the invention under the culturing conditions.

In one embodiment of the present invention, the host cell is a mammalian cell. Mammalian cells are particularly suitable hosts for production of glycoproteins, due to their capability to glycosylate proteins in the most compatible form for human application (Cumming et al., Glycobiology 1: 115-30 (1991); Jenkins et al., Nature Biotechnol. 14:975-81 (1996)).

In one embodiment of the present invention, the mammalian cell is a CHO cell, cell line CHO-K1 (ATCC CCL-61), cell line DUXB11 (ATCC CRL-9096) and cell line Pro-5 (ATCC CRL-1781) registered at ATCC, commercially available cell line CHO-S (Cat #11619 of Life Technologies)), a BHK cell (including the commercially available cell line ATCC accession no. CCL 10), a NSO cell, NSO cell line (RCB 0213) registered at RIKEN Cell Bank, The Institute of Physical and Chemical Research, subcell lines obtained by naturalizing these cell lines to media in which they can grow, and the like), a SP2/0 cell, a SP2/0-Ag14 cell, SP2/0-Ag14 cell (ATCC CRL-1581) registered at ATCC, subcell lines obtained by naturalizing these cell lines to media in which they can grow (ATCC CRL-1581.1), and the like), a YB2/0 cell, a PER cell, a PER.C6 cell, subcell lines obtained by naturalizing these cell lines to media in which they can grow, and the like, a rat myeloma cell line YB2/3HL.P2.G11.16Ag.20 cell (including cell lines established from Y3/Ag1.2.3 cell (ATCC CRL-1631), YB2/3HL.P2.G11.16Ag.20 cell, YB2/3HL.P2.G11.16Ag.20 cell (ATCC CRL-1662) registered at ATCC, sub-lines obtained by naturalizing these cell lines to media in which they can grow, and the like), a hybridoma cell, a human leukemic Namalwa cell, an embryonic stem cell, or a fertilized egg cell.

In one embodiment of the present invention, the activity of mannosidase II in the host cell is decreased by addition of a mannosidase II inhibitor. Mannosidase II (EC 3.2.1.114) refers to a mannosyl-oligosaccharide 1,3-1,6-alpha-mannosidase enzyme which hydrolyses the terminal (1->3)- and (1->6)-linked alpha-D-mannose residues in the mannosyl-oligosaccharide GlcNAcMan5GlcNAc2. In one embodiment of the invention, the mannosidase II enzyme is a mammalian enzyme. Examples of mannosidase II enzymes include human mannosidase II A1 (MAN2A1; Gene ID: 4124; Accession No. NM_{—}002372, protein: NP_{—}002363.2 (SEQ ID NO: 1)), human mannosidase II A2 (MAN2A2; Gene ID: 4122; Accession No. NM_{—}006122, protein NP_{—}006113 (SEQ ID NO: 2)), mouse MAN2A1 (Accession No. NM_{—}008549, protein NP_{—}032575.2 (SEQ ID NO: 3)), mouse MAN2A2 (Accession No. NM_{—}172903, protein NP_{—}766491.2 (SEQ ID NO: 4)), rat MAN2A1 (Accession No. NM_{—}012979, protein NP_{—}037111.2 (SEQ ID NO:5)), and rat MAN2A2 (Accession No. NM_{—}001107527, protein NP_{—}001100997.1 (SEQ ID NO: 6)).

In one embodiment of the present invention, the mannosidase II inhibitor is swainsonine.

In one embodiment of the present invention, the activity of mannosidase II or GnTII in the host cell is decreased by RNA interference (RNAi). RNAi refers to the introduction of homologous double stranded RNA to specifically target the transcription product of a gene, resulting in a null or hypomorphic phenotype. RNA interference requires an initiation step and an effector step. In the first step, input double-stranded (ds) RNA is processed into nucleotide ‘guide sequences’. These may be single- or double-stranded. The guide RNAs are incorporated into a nuclease complex, called the RNA-induced silencing complex (RISC), which acts in the second effector step to destroy mRNAs that are recognized by the guide RNAs through base-pairing interactions. RNAI molecules are thus double stranded RNAs (dsRNAs) that are very potent in silencing the expression of the target gene. The invention provides dsRNAs complementary to the mannosidase II and GnTII gene.

The ability of dsRNA to suppress the expression of a mannosidase II or a GnTII gene corresponding to its own sequence is also called post-transcriptional gene silencing or PTGS. The only RNA molecules normally found in the cytoplasm of a cell are molecules of single-stranded mRNA. If the cell finds molecules of double-stranded RNA, dsRNA, it uses an enzyme to cut them into fragments containing in general 21-base pairs (about 2 turns of a double helix). The two strands of each fragment then separate enough to expose the antisense strand so that it can bind to the complementary sense sequence on a molecule of mRNA. This triggers cutting the mRNA in that region thus destroying its ability to be translated into a polypeptide. Introducing dsRNA corresponding to a particular gene will knock out the cell's endogenous expression of that gene. A possible disadvantage of simply introducing dsRNA fragments into a cell is that gene expression is only temporarily reduced. However, a more permanent solution is provided by introducing into the cells a DNA vector that can continuously synthesize a dsRNA corresponding to the gene to be suppressed.

RNAi molecules are prepared by methods well known to the person skilled in the art. In general, an isolated nucleic acid sequence comprising a nucleotide sequence which is substantially homologous to the sequence of at least one of the mannosidase II genes or one of the GnTII genes and which is capable of forming one or more transcripts able to form a partially of fully double stranded (ds) RNA with (part of) the transcription product of said mannosidase II genes or GnTII genes will function as an RNAi molecule. The double stranded region may be in the order of between 10-250, preferably 10-100, more preferably 20-50 nucleotides in length.

RNA interference (RNAi) is a method for regulating gene expression. For example, double-stranded RNA complementary to mannosidase II or GnTII can decrease the amount of this glycosyltransferase expressed in an antibody expressing cell line, resulting in an increased level of glycoprotein of the invention. Unlike in gene knockouts, where the level of expression of the targeted gene is reduced to zero, by using different fragments of the particular gene, the amount of inhibition can vary, and a particular fragment may be employed to produce an optimal amount of the desired glycoprotein or composition thereof. An optimal level can be determined by methods well known in the art, including in vivo and in vitro assays for Fc receptor binding, effector function including ADCC, efficacy, and toxicity. The use of the RNAi knockdown approach, rather than a complete knockout, allows the fine tuning of the amount of glycan structures according to the invention to an optimal level, which may be of great benefit, if the production of glycoproteins bearing less than 100% of oligosaccharides according to Formula I is desirable.

In one embodiment of the present invention, the activity of mannosidase II in the host cell is decreased by gene disruption (knockout) of all necessary genes encoding mannosidase II isoforms in the host cell, such as MAN2A1 (mannosidase II) and MAN2A2 (mannosidase IIx) in a human cell. A person skilled in the art can identify mannosidase II genes in the host cell based on e.g. sequence similarity to the human genes.

In one embodiment of the present invention, the host cell has reduced activity of GnTII compared to the parent cell. “Activity of GnTII” should be understood as meaning correlation between a level of GnTII enzyme activity to transfer a GlcNAc residue to the oligosaccharide structure according to Formula I attached to the glycoprotein of the invention and % portion of the GlcNAc's transferred to the oligosaccharide structures according to formula I attached to glycoproteins in the composition of the invention. A host cell has “reduced or decreased activity of GnTII” when said cell produces lower % portion of the GlcNAc's transferred to the oligosaccharide structures according to formula I attached to glycoproteins in the composition of the invention compared to parent cell without manipulations to decrease GnTII activity when cultured in similar or identical conditions.

“GnTII” refers to mannosyl (alpha-1,6-)glycoproteinbeta-1,2-N-acetylglucosaminyltransferase. The protein is a Golgi enzyme catalyzing an essential step in the conversion of oligomannose to complex N-glycans. In one embodiment of the present invention, GnTII is a mammalian enzyme. Examples of GnTII enzymes include human GnTII (Gene ID: 4247; Accession Nos. NM_{—}001015883, NM_{—}002408, NP_{—}001015883 and NP_{—}002399 (SEQ ID NO: 7)), rat GnTII (GeneID: 94273 Accession Nos. NM_{—}053604 and NP_{—}446056 (SEQ ID NO: 8)), mouse (Accession No. NM_{—}146035; protein Accession No. NP_{—}666147 (SEQ ID NO: 9)), and Chinese hamster (Accession No. XM_{—}003513994; protein Accession No. XP_{—}003514042 (SEQ ID NO: 10); from CHO-K1 cells). The term “GNTII” refers to a gene or polynucleotide encoding a GnTII enzyme, including the coding region, noncoding region preceding (leader) and following coding regions, introns, and exons of a GNTII sequence. In particular, the GNTII gene includes the promoter. In one embodiment of the present invention, the activity of GnTII in the host cell is decreased by RNA interference (RNAi).

In one embodiment of the present invention, the activity of GnTII in the host cell is decreased by gene disruption (knockout). A person skilled in the art can identify the GnTII gene in the host cell based on e.g. sequence similarity to the human gene.

In this context, the term “parent cell” should be understood as meaning a host cell before decreasing or deleting activity of mannosidase II or GnTII in said cell.

In one embodiment of the present invention, the host cell further has increased activity of N-glycan β1,4-galactosylation and sialylation.

In one embodiment of the present invention, the host cell comprising a polynucleotide encoding the protein moiety of a glycoprotein of the invention further has

a) reduced activity of mannosidase II or GnTII, and

b) optimized, or increased, activity of β4-galactosyltransferase and/or α2,3/6-sialyltransferase

compared to the parent cell.

In one embodiment of the present invention, the host cell further has increased activity of core fucosylation compared to the parent cell.

In one embodiment of the present invention, the host cell has increased activity of α2,6-sialyltransferase compared to the parent cell.

The present invention further relates to a host cell comprising a polynucleotide encoding the protein moiety of a glycoprotein according to the invention, wherein said host cell has increased activity of core fucosylation compared to the parent cell. In this context, the term “core fucosylation” should be understood as meaning any enzymatic activity capable of biosynthesis of GDP-fucose or of adding a Fuc residue to the core GlcNAc residue present in the core Manβ4GlcNAcβ4GlcNAc N-glycan structure that is linked by a β-N linkage to Asn, or proteins needed for intracellular transport or GDP-fucose. In this context “increased activity of core fucosylation” or “the activity of core fucosylation is increased” means herein any method which results increase of core fucosylation of glycoproteins of the invention, preferably in a host cell. A host cell has “increased activity of core fucosylation” or “the activity of core fucosylation increased” when said cell produces higher % portion of the fucose residues in the oligosaccharide structures according to Formula I attached to glycoproteins in the composition of the invention compared to parent cell without manipulations to increase the activity of core fucosylation when cultured in similar or identical conditions. Increased activity of core fucosylation in a host cell is also achieved by increasing the activity of an enzyme relating to the synthesis of an intracellular sugar nucleotide, GDP-fucose. The enzymes include GMD (GDP-mannose 4,6-dehydratase); (b) Fx (GDP-keto-6-deoxymannose 3,5-epimerase, 4-reductase); (c) GFPP (GDP-beta-L-fucose pyrophosphorylase). Increase of core fucosylation can also be achieved by increasing the activity of α-1,6-fucosyltransferase or FUT8. As the method for obtaining such cells, any technique can be used, so long as it can increase the activity of core fucosylation. In one embodiment that may be combined with the preceding and following embodiments, the host cell has increased activity of core fucosylation compared to parent cell.

The present invention further relates to a method for producing the glycoprotein according to the invention comprising the step of

a) culturing the host cell comprising a polynucleotide encoding the protein moiety of a glycoprotein according to the invention in the presence of mannosidase II inhibitor.

The present invention further relates to a method for producing the composition according to the present invention, characterised in that it comprises the steps of

a) culturing a host cell comprising a polynucleotide encoding the protein moiety of a glycoprotein of the invention in the presence of mannosidase II inhibitor; or the steps of

a′) culturing a host cell according to the present invention; and

a″) recovering the glycoprotein composition from the host cell culture.

In one embodiment of the present invention, the method further comprises the steps of

b) contacting the product of step a), a′), or a″) with an β1,4-galactosyltransferase in the presence of UDP-Gal; and/or

c) contacting the product of step b) with a α2,6-sialyltransferase in the presence of CMP-NeuNAc.

In one embodiment of the present invention, the method further comprises the steps of

b) contacting the product of step a), a′), or a″) with an β1,4-galactosyltransferase in the presence of UDP-Gal to produce a glycoprotein comprising a hybrid-type oligosaccharide structure comprising a terminal Gal residue; and/or
c) contacting the product of step b) with a α2,6-sialyltransferase in the presence of CMP-NeuNAc.

Since the product of step a) is typically a mixture of glycoforms comprising the oligosaccharide structure according to the invention together with other glycoforms comprising related (sharing a common structural feature) oligosaccharide structures, steps b) and c) of this embodiment lead to an increased yield of the glycoprotein according to the invention.

The present invention further relates to a method for producing the composition according to the present invention, wherein the method comprises the steps of

a′) culturing a host cell according to any one of claims 16-19; and

a″) recovering the glycoprotein composition from the host cell culture.

The present invention further relates to a method for producing the composition according to the present invention, characterised in that it comprises the steps of

a) culturing a host cell comprising a polynucleotide encoding the protein moiety of a glycoprotein of the invention in the presence of mannosidase II inhibitor.

In one embodiment of the present invention, the method further comprises the step of contacting the product of the previous step with α-mannosidase. This embodiment leads to the predominant production of the glycoprotein according to formula I wherein x=0 and y=0.

In one embodiment of the present invention, the host cell is cultured in the presence of swainsonine in a concentration of at least 60 μM.

In one embodiment of the present invention, the host cell is cultured in the presence of swainsonine in a concentration of at least 100 μM. In one embodiment of the present invention, the host cell is manipulated to express optimized levels of a β4-galactosyltransferase and/or an α2,3/6-sialyltransferase activity to generate glycoprotein composition of the invention. In one embodiment, the host cell is selected for the optimized level of a β4-galactosyltransferase and/or a α2,3/6-sialyltransferase activity to generate glycoprotein composition of the invention. In one embodiment, the host cell is manipulated to increase the activity of a β4-galactosyltransferase and/or a α2,3/6-sialyltransferase compared to parent cell to generate glycoprotein composition of the invention.

Specifically, such host cell may be manipulated to comprise a recombinant nucleic acid molecule encoding a β4-galactosyltransferase and/or a α2,3/6-sialyltransferase, operatively linked to a constitutive or regulated promoter system. In one embodiment, the host cell is transformed or transfected with a nucleic acid molecule comprising a gene encoding a β4-galactosyltransferase and/or with a nucleic acid molecule comprising a gene encoding a α2,3/6-sialyltransferase. In one embodiment, the host cell is manipulated such that an endogenous β4-galactosyltransferase and/or α2,3/6-sialyltransferase has been activated by insertion of a regulated promoter element into the host cell chromosome. In one embodiment, the host cell has been manipulated such that an endogenous β4-galactosyltransferase and/or α2,3/6-sialyltransferase has been activated by insertion of a constitutive promoter element, a transposon, or a retroviral element into the host cell chromosome.

Alternatively, a host cell may be employed that naturally produce, are induced to produce, and/or are selected to produce a β4-galactosyltransferase and/or a α2,3/6-sialyltransferase. In one embodiment, the host cell has been selected in such way that an endogenous β4-galactosyltransferase and/or α2,3/6-sialyltransferase is activated. For example, the host cell may be selected to carry a mutation triggering expression of an endogenous β4-galactosyltransferase and/or α2,3/6-sialyltransferase.

In one embodiment, the activity of a β4-galactosyltransferase and/or a α2,3/6-sialyltransferase in the host cell is increased compared to the parent cell to generate glycoprotein composition of the invention. In this context, the term “parent cell” should be understood as meaning a host cell before increasing activity of a β4-galactosyltransferase and/or a α2,3/6-sialyltransferase in said cell.

“Activity of β4-galactosyltransferase” or “levels of β4-galactosyltransferase activity” should be understood as meaning correlation between a level of β4-galactosyltransferase enzyme activity to transfer a Gal residue in the oligosaccharide structure according to Formula I-III attached to the glycoprotein of the invention and % portion of the galactose residues in the oligosaccharide structures according to formula I attached to glycoproteins in the composition of the invention. A host cell has “increased activity of β4-galactosyltransferase” when said cell produces higher % portion of the galactose residues in the oligosaccharide structures according to formula I attached to glycoproteins in the composition of the invention compared to parent cell without manipulations to increase β4-galactosyltransferase activity when cultured in similar or identical conditions. A host cell has “optimized activity of β4-galactosyltransferase” when said cell produces higher or lower % portion of the galactose residues in the oligosaccharide structures according to formula I attacked to glycoproteins in the composition of the invention compared to parent cell without manipulations to optimize β4-galactosyltransferase activity when cultured in similar or identical conditions. Optimal levels of β4-galactosyltransferase activity in a host cell depend on % portion of the galactose residues in the oligosaccharide structures according to formula I attached to glycoproteins in the composition of the invention. Typically, host cell is manipulated to have increased levels of β4-galactosyltransferase activity compared to parent cell when cultured in similar or identical conditions.

“Activity of α2,3/6-sialyltransferase” or “level of α2,3/6-sialyltransferase activity” should be understood as meaning correlation between a level of α2,3/6-sialyltransferase enzyme activity to transfer a Neu5Ac residue in the oligosaccharide structure according to Formula I attached to the glycoprotein of the invention and % portion of the Neu5Ac residues in the oligosaccharide structures according to Formula I attached to glycoproteins in the composition of the invention. A host cell has “increased activity of α2,3/6-sialyltransferase” or “increased level α2,3/6-sialyltransferase of activity” when said cell produces higher % portion of the Neu5Ac residues in the oligosaccharide structures according to formula I attached to glycoproteins in the composition of the invention compared to parent cell without manipulations to increase α2,3/6-sialyltransferase activity when cultured in similar or identical conditions. A host cell has “optimized activity of α2,3/6-sialyltransferase” when said cell produces higher or lower % portion of the Neu5Ac residues in the oligosaccharide structures according to formula I attached to glycoproteins in the composition of the invention compared to parent cell without manipulations to optimize α2,3/6-sialyltransferase activity when cultured in similar or identical conditions. Optimal levels of α2,3/6-sialyltransferase activity in a host cell depend on % portion of the Neu5Acα2,3/6 residues in the oligosaccharide structures according to formula I attached to glycoproteins in the composition of the invention. A host cell may be manipulated to have increased levels of α2,6-sialyltransferase activity compared to parent cell when cultured in similar or identical conditions, thus, host cell produces increased % portion of Neu5Ac residues in the oligosaccharide structures according to formula I attached to glycoproteins in the composition of the invention wherein Z=6.

A host cell has “decreased or reduced activity of α2,3-sialyltransferase” or “decreased or reduced level of α2,3-sialyltransferase activity” when said cell produces lower % portion of the Neu5Acα2,3 residues in the oligosaccharide structures according to formula I attached to glycoproteins in the composition of the invention compared to parent cell without manipulations to decrease or reduce activity of α2,3-sialyltransferase when cultured in similar or identical conditions. In a host cell decreased level of α2,3-sialyltransferase activity may results increased levels of α2,6-sialyltransferase activity and/or higher % portion of the Neu5Acα2,6 residues in the oligosaccharide structures according to formula I attached to glycoproteins in the composition of the invention.

In one embodiment, the activity of mannosidase II in the host cell is decreased and the levels of a β4-galactosyltransferase and a α2,3/6-sialyltransferase activities are optimized or increased in said cell compared to parent cell.

In one embodiment, the activity of GnTII in the host cell is decreased and the levels of a β4-galactosyltransferase and a α2,3/6-sialyltransferase activities are optimized or increased in said cell compared to parent cell.

In one embodiment, the host cell is manipulated to express optimized levels of a β4-galactosyltransferase and a α2,3/6-sialyltransferase activity, and the activity of mannosidase II or GnTII in said cell is decreased compared to parent cell, to generate the glycoprotein composition of the invention.

In one embodiment, the host cell is manipulated to express optimized levels of a β4-galactosyltransferase and the activity of mannosidase II in the said cell is decreased compared to parent cell, to generate the glycoprotein composition of the invention.

In one embodiment that may be combined with the preceding embodiments, the host cell is essentially devoid of the activity of mannosidase II or GnTII.

In one embodiment, the host cell is manipulated to express increased levels of a β4-galactosyltransferase activity, increased levels of a α2,6-sialyltransferase activity and decreased levels of a α2,3-sialyltransferase activity, and the activity of mannosidase II or GnTII in said cell is decreased compared to parent cell, to generate the glycoprotein or the composition of the invention. The enzyme β1,4-galactosyltransferase adds the Gal residue present in the oligosaccharide structure according to formula I. In one embodiment, β4-galactosyltransferase is a mammalian enzyme. In one embodiment of the present invention, the β1,4-galactosyltransferase is bovine milk β1,4-galactosyltransferase or human β1,4-galactosyltransferase I (GenBank Accession No. P15291; SEQ ID NO: 11). Examples of β4-galactosyltransferase include but are not limited to rat β4-galactosyltransferase (GenBank Accession No. NP_{—}445739; SEQ ID NO: 12), mouse β4-galactosyltransferase (GenBank Accession No. P15535; SEQ ID NO: 13), and Chinese hamster β4-galactosyltransferase I (GenBank Accession No. NP_{—}001233620; SEQ ID NO: 14). Other β4-galactosyltransferases include human B4GALT2 (GenBank Accession No. O60909), human B4GALT3 (GenBank Accession No. O60512), human B4GALT4 GenBank Accession No. O60513), and human B4GALT5 GenBank Accession No. O43286) and their homologues in mouse, rat, and Chinese hamster.

The enzyme α2,6-sialyltransferase adds the terminal Neu5Ac residue present in the oligosaccharide structure according to formula I. In one embodiment, the α2,6-sialyltransferase is a mammalian enzyme. In one embodiment of the present invention, the α2,6-sialyltransferase is a rat recombinant α2,6-sialyltransferase (GenBank accession No. P13721; SEQ ID NO: 15; GenBank accession No. Q701R3; SEQ ID NO: 16), a rat liver α2,6-sialyltransferase, human α2,6-sialyltransferase I (GenBank accession No. P15907; SEQ ID NO: 17) or human α2,6-sialyltransferase II (GenBank accession No. Q96JF0; SEQ ID NO: 18), mouse α2,6-sialyltransferase (GenBank accession No. NP_{—}666045; SEQ ID NO: 19 and GenBank accession No. Q76K27; SEQ ID NO: 20) and Chinese hamster α2,6-sialyltransferase (GenBank accession No. NP_{—}001233744; SEQ ID NO: 21 and GenBank accession No. XP_{—}003499570; SEQ ID NO: 22).

In one embodiment, the α2,3-sialyltransferase is a mammalian enzyme. In one embodiment of the present invention, the α2,3-sialyltransferase is a human ST3GAL2, ST3GAL4 and ST3GAL6 enzyme (GenBank accession No. Q16842, SEQ ID NO: 23; GenBank accession No. Q11206, SEQ ID NO: 24; and GenBank accession No. Q9Y274, SEQ ID NO: 25) or their isoforms. In one embodiment of the present invention, the α2,3-sialyltransferase is a rat α2,3-sialyltransferase (GenBank accession Nos. Q11205, P61131, and P61943), mouse α2,3-sialyltransferase (GenBank accession Nos. Q11204, Q91Y74, and Q8VIB3) or Chinese hamster α2,3-sialyltransferase (GenBank accession Nos. NP_{—}001233628, and XP_{—}003509939).

In one embodiment of the present invention, the host cell further has decreased activity of a sialidase compared to the parent cell.

In one embodiment of the present invention, activity of a sialidase, especially a cytosolic sialidase activity is decreased or abolished in the host cell compared to the parent cell. In one embodiment of the present invention, a host cell expressing β4-galactosyltransferase and/or α2,3/6-sialyltransferase is selected so that activity of a sialidase activity is decreased or abolished, the level of activity of a sialidase produced by the host cell being such that sialic acid residues in the carbohydrate side-chains of glycoprotein produced by the host cell are not cleaved, or are not cleaved to an extent which affects the function of the glycoprotein. In one embodiment, activity of sialidase activity is reduced using RNAi. In one embodiment, activity of sialidase activity is decreased by gene knock-out.

In one embodiment, heterogeneity of glycoprotein composition of the present invention is reduced by expressing optimized levels of a β4-galactosyltransferase activity and/or a α2,3/6-sialyltransferase activity in the host cell. In one embodiment, heterogeneity of glycoprotein composition of the present invention is reduced by decreasing the activity of one α2,3/6-sialyltransferase and increasing the activity of the other α2,3/6-sialyltransferase in the host cell compared to the parent cell. In some embodiments, the activity of α2,3-sialyltransferase is decreased in the host cell compared to the parent cell. In some embodiments, the activity of α2,3-sialyltransferase is decreased and the activity of α2,6-sialyltransferase is increased in the host cell compared to the parent cell.

For example, in the case of CHO cells it is known that CHO derived recombinant glycoproteins have exclusively α-2,3-linked sialic acids, since the CHO genome does not include a gene which codes for a functional α2,6-sialyltransferase. If a glycoprotein composition of the present invention is desired to be produced in the CHO cell, the activity of mannosidase II is decreased and the level of a β4-galactosyltransferase activity and/or the level of an α2,3-sialyltransferase activity are optimized or increased in the said CHO cell. In one embodiment, the activity of GnTII in the CHO cell is decreased, the level of a β4-galactosyltransferase activity and/or the level of an α2,3-sialyltransferase activity are optimized or increased in said CHO cell.

If a glycoprotein composition of the present invention is desired to be produced in CHO cells and glycoprotein composition is desired to comprise α-2,6-linked sialic acids, in one embodiment, the activity of mannosidase II is decreased, the activity of β4-galactosyltransferase is increased or optimized, and the activity of α2,6-sialyltransferase is increased and/or optimized in said CHO cell compared to the parent cell. In one embodiment, the activity of a GnTII in the CHO cell is decreased and the activity of a β4-galactosyltransferase and the activity of an α2,6-sialyltransferase are increased and/or optimized compared to parent cell. In one embodiment that may be combined with the preceding embodiments the CHO cell is essentially devoid of the activity of a GnTII. In one embodiment that may be combined with the preceding embodiments the CHO cell is essentially devoid of the activity of an α2,3-sialyltransferase.

Methods which are well known to those skilled in the art can be used to construct expression vectors containing the polynucleotide encoding the protein moiety of a glycoprotein according to the invention, the coding sequence of a β4-galactosyltransferase and/or a α2,3/6-sialyltransferase, appropriate transcriptional/translational control signals, possible use of reporter genes as well as a mannosidase II, a GnTII, and a α2,3/6-sialyltransferase, such as α2,3-sialyltransferase, knock-out deletion or RNAi constructs. The methods include in vitro recombinant DNA techniques, synthetic techniques and in vivo recombination/genetic recombination.

Methods which are well known to those skilled in the art can be used to express a polynucleotide encoding the protein moiety of a glycoprotein according to the invention, nucleic acids encoding a β4-galactosyltransferase, a α2,3/6-sialyltransferase, and above deletion and RNAi constructs in a host cell. Nucleic acids may be expressed under the control constitutive promoters or using regulated expression systems such as a tetracycline-regulated expression system, a lac-switch expression system, and a metallothionein metal-inducible expression system. If nucleic acids encoding a β4-galactosyltransferase and a α2,3/6-sialyltransferase are comprised within the host cell, one of them may be expressed under the control of a constitutive promoter, while other is expressed under the control of a regulated promoter. The optimal expression levels will be different for each protein of interest, and will be determined using routine experimentation. Expression levels are determined by methods generally known in the art, including Western blot analysis using a glycosyl transferase or a glycosyl hydrolase specific antibody, protein tag specific antibodies, Northern blot analysis using a polynucleotide encoding the protein moiety of a glycoprotein according to the invention, a glycosyl transferase or glycosyl hydrolase specific nucleic acid probe, or measurement of enzymatic activity. Alternatively, a lectin may be employed which binds to glycans produced by the glycosyl transferases or glycosyl hydrolases, for example, agglutinins from Erythrina cristagalli (ECA) and Ricinus communis (RCA) binding to Galβ1-4GlcNAc, Sambucus nigra (SNA) binding to α2,6-linked sialic acid, Maackia amurensis (MAA) binding to α2,3-linked sialic acid, Galanthus nivalis (GNA) and Hippeastrum hybrid (HHA) binding to α-mannose, Lens culinaris (LCA) binding to N-glycan core α1,6-linked fucose, and the like.

For the methods of this invention, stable expression is generally preferred to transient expression and also is more amenable to large scale production. Rather than using expression vectors which contain viral origins of replication, host cells can be transformed with the respective coding nucleic acids controlled by appropriate expression control elements and a selectable marker. Following the introduction of foreign DNA, a number of selection systems may be used, which are well known to those skilled in the art.

The host cell comprising a polynucleotide encoding the protein moiety of a glycoprotein according to the invention or the host cell producing the glycoprotein composition of the present invention may be identified, for example, by detection by immunoassay, by its biological activity, or by mass spectrometric means described below.

The glycoprotein or the glycoprotein composition produced by the host cell of the invention can be assessed immunologically, for example by Western blots, immunoassays such as radioimmuno-precipitation, enzyme-linked immunoassays and the like. In one embodiment, glycoprotein composition is assayed in in vitro or in vivo tests, for example, as described in Examples. The present invention provides host cells for the producing composition comprising a glycoprotein comprising the Fc domain of an antibody, or a fragment thereof, comprising an Asn residue and an oligosaccharide structure attached thereto, and that the oligosaccharide structure has a structure according to formula I. Generally, the host cell has been transformed to express nucleic acids encoding the protein moiety of the glycoprotein for which the production of glycoforms according to Formula I-III are desired, along with at least one nucleic acid encoding a RNAi, knock-out, or any other construct meant for decreasing the activity of mannosidase II, GnTII, sialidase or α2,3/6-sialyltransferase, or nucleic acids encoding a β4-galactosyltransferase or α2,3/6-sialyltransferase to increase the activity of β4-galactosyltransferase and/or α2,3/6-sialyltransferase. Typically, the transfected cells are selected to identify and isolate clones that express the any of the above nucleic acids including mannosidase II, GnTII, β4-galactosyltransferase, and α2,3/6-sialyltransferase as well as nucleic acids encoding the protein moiety of the glycoprotein. Transfected cells may be assayed with methods described above and Examples to identify and select host cells having optimized levels of β4-galactosyltransferase activity and/or α2,3/6-sialyltransferase activity as well as decreased mannosidase II or GnTII activity. Host cells transfected with nucleic acids encoding the protein moiety of the glycoprotein and cultured under conditions suitable for expression of the protein moiety of the glycoprotein may be assayed with methods described above and Examples to identify and select host cells having optimized levels of β4-galactosyltransferase activity and/or α2,3/6-sialyltransferase and decreased mannosidase II or GnTII activity. In one embodiment, the host cell has been selected for expression of endogenous β4-galactosyltransferase, α2,3/6-sialyltransferase, mannosidase II and/or GnTII activity.

For example, host cells may be selected carrying mutations which trigger expression of otherwise silent β4-galactosyltransferase activity and/or α2,3/6-sialyltransferase activity. For example, host cells may be selected carrying mutations which inactivate expression of otherwise active mannosidase II or GnTII activity.

In one embodiment of the present invention, a method for the producing composition of the invention comprises the steps of a) transforming a host cell with vectors or constructs comprising nucleic acid molecules encoding a protein moiety of the glycoprotein of the invention, b) transforming the host cell with vectors or constructs comprising nucleic acid molecules reducing the activity of mannosidase II or GnTII activity, or culturing said cells in the presence of mannosidase II inhibitor, c) transforming the host cell with vectors or constructs comprising nucleic acid molecules encoding optimized levels of β4-galactosyltransferase activity and/or optimized levels of α2,3/6-sialyltransferase activity, d) culturing the host cell under conditions that allow synthesis of said protein moiety of the glycoprotein and gene products of steps b) and c); and e) recovering said glycoprotein composition from said culture.

The method according to the invention may further comprise the step of recovering the glycoprotein from cell culture or from a reaction mixture. The glycoprotein composition may be recovered as crude, partially purified or highly purified fractions using any of the well-known techniques for obtaining glycoprotein from cell cultures. This step may be performed by e.g. precipitation, purification by using techniques such as lectin chromatography or contacting the glycoprotein with immobilized Fc receptor, carbohydrate-binding protein or protein G or A, or any other method that produces a preparation suitable for further use.

In one embodiment of the present invention, the method further comprises the step of recovering the glycoprotein composition, and adding a pharmaceutically acceptable carrier.

The methods of producing the glycoprotein according to the invention usually produce a mixture of glycoforms, i.e. a mixture of glycoforms comprising the oligosaccharide structure according to the invention together with other glycoforms comprising related (sharing a common structural feature) oligosaccharide structures. Therefore the present invention further relates to a method for producing the composition according to the invention comprising the step of

a) culturing the host cell comprising a polynucleotide encoding the protein moiety of a glycoprotein according to the invention in the presence of mannosidase II inhibitor.

In one embodiment of the present invention, the method further comprises the steps of

b) contacting the product of step a) with an β1,4-galactosyltransferase in the presence of UDP-Gal; and
c) contacting the product of step b) with a α2,6-sialyltransferase in the presence of CMP-NeuNAc.

The method according to the invention may further comprise the step of adding a pharmaceutical carrier or any other ingredients suitable for a pharmaceutical composition.

In one embodiment of the present invention, the method for producing the glycoprotein according to the invention or the composition according to the invention comprises the step of a) culturing a host cell according to the invention.

The present invention further relates to a host cell comprising a polynucleotide encoding the protein moiety of a glycoprotein according to the invention, wherein said host cell has reduced activity of mannosidase II or GnTII and optimized, or increased, levels of a β4-galactosyltransferase activity and a α2,3/6-sialyltransferase activity compared to the parent cell.

The present invention further relates to a method for producing the glycoprotein according to the invention comprising the step of a) culturing the host cell comprising a polynucleotide encoding the protein moiety of a glycoprotein according to the invention and which cell has optimized or increased levels of a β4-galactosyltransferase activity and a α2,3/6-sialyltransferase activity compared to the parent cell in the presence of mannosidase II inhibitor.

The present invention further relates to a host cell comprising a polynucleotide encoding the protein moiety of a glycoprotein according to the invention, wherein said host cell has reduced activity of mannosidase II or GnTII, optimized, or increased, activity of a β4-galactosyltransferase, increased activity of an α2,6-sialyltransferase, and reduced, or abolished, activity of an α2,3-sialyltransferase compared to the parent cell.

The glycoprotein or glycoprotein composition of any above step may be contacted in vitro with β4-galactosyltransferase in the presence of UDP-Gal, with a α2,6-sialyltransferase in the presence of CMP-NeuNAc and/or with an α-mannosidase.

The present invention further relates to a method of treating autoimmune diseases, inflammatory disorders or any other disease where binding to an antibody target or increased anti-inflammatory activity with reduced cytotoxic activity is desired, wherein the glycoprotein or composition according to the invention is administered to a human or animal in an effective amount. The effective amount may vary depending on a number of factors and may be determined depending on e.g. the patient's age, size, the nature of the glycoprotein, and the administration route.

In this context, the term “treatment” should be understood as the administration of an effective amount of a therapeutically active compound of the present invention with the purpose of easing, ameliorating, alleviating, inhibiting, slowing down progression, or reduction of disease burden or eradicating (curing) symptoms of the disease or disorder in question. In one embodiment of the present invention, the term “treatment” should also be understood as meaning a prophylactive therapy meaning preventative therapy without meaning an absolute prevention or cure, but reduction of occurrence, or alleviation, inhibition, slowing down progression of the disease, or reduction of disease burden in the future partially in a patient.

The embodiments of the invention described hereinbefore may be used in any combination with each other. Several of the embodiments may be combined together to form a further embodiment of the invention. A product, or a use, or a method to which the invention is related, may comprise at least one of the embodiments of the invention described hereinbefore.

The glycoprotein of the invention has a number of advantages over glycoproteins comprising other oligosaccharide structures typically attached to said glycoproteins, such as normal oligosaccharide structures. The presence of the fucose residue and the sialic acid residue in the oligosaccharide structure according to the invention greatly decrease the cytotoxicity of the glycoprotein and increase anti-inflammatory activity. The invention therefore provides glycoproteins that may be highly effective for treating pathologies wherein a reduction of inflammatory activity is desired. Furthermore, the presence of non-reducing terminal Man residues in the α6 branch of the oligosaccharide structure leads to improved fucosylation, galactosylation and sialylation (addition of Fuc, Gal and Neu5Ac into the oligosaccharide structure according to formula I) when the glycoprotein of the invention is produced in a mammalian host cell.

### Examples

In the following, the present invention will be described in more detail. Reference will now be made in detail to the embodiments of the present invention, examples of which are illustrated in the accompanying drawings. The description below discloses some embodiments of the invention in such detail that a person skilled in the art is able to utilize the invention based on the disclosure. Not all steps of the embodiments are discussed in detail, as many of the steps will be obvious for the person skilled in the art based on this specification.

### Example 1

### Production of Humanized IgG1 Antibody Glycoforms in CHO Cells

Humanized anti-IL-8 IgG1 antibody producing cell line DP-12 (ATCC number CRL-12445) was grown in DMEM with 4 mM L-glutamine and adjusted with sodium bicarbonate and 4.5 g/L glucose and 200 nM methotrexate, trace elements A and B from Mediatech, 0.002 mg/ml rhInsulin and 10% fetal bovine serum. For antibody production, cells were grown for 3-4 days and the supernatant collected by centrifugation.

Glycosidase inhibitors were added to the culture medium to produce hybrid-type antibody glycoforms: 10 μg/ml swainsonine (Cayman Chemical).

Antibody glycoforms were purified from cell culture supernatants by protein G affinity chromatography on a 1-mL HiTrap protein G column (GE Healthcare, Uppsala, Sweden) using single step pH gradient elution from 20 mM sodium phosphate, pH 7.0 to 0.1 M citric acid, pH 2.6. The eluted antibody fractions were neutralized immediately with 1 M Na2HPO4 and concentrated in Millipore Amicon Ultracel 30K concentrators. The concentrations of antibody glycoforms were adjusted to 0.5 mg/ml with phosphate-neutralized 0.1 M citric acid.

### Mass Spectrometric Analysis of Antibody Glycoforms

For N-glycan analysis antibody solution containing 10-20 μg antibody was applied to N-glycan release; optionally antibodies were first precipitated with 67% (v/v) ice-cold ethanol and pelleted by centrifugation; cells were collected, washed repeatedly with phosphate buffered saline and pelleted by centrifugation.

N-glycan release, purification for analysis, permethylation and MALDI-TOF mass spectrometric fragmentation analysis were performed essentially as described previously (Satomaa et al., Cancer Research 2009, 69, 5811-5819) with minor modifications. N-linked glycans were detached by enzymatic hydrolysis with N-glycosidase F (Glyko). N-glycans were first purified on Hypersep C-18 and then on Hypersep Hypercarb 50 mg 96-well plates (Thermo Scientific). The neutral and acidic N-glycans were eluted together from Hypercarb with 0.05% trifluoroacetic acid in 25% acetonitrile in water. Matrix-assisted laser desorption-ionization time-of-light (MALDI-TOF) mass spectrometry was performed with a Bruker Ultraflex III instrument (Bruker Daltonics, Germany). Neutral and acidic N-glycans were detected in positive ion reflector mode as sodium adduct ions using 2,5-dihydroxybenzoic acid (DHB, Aldrich) as the matrix. Each of the steps in the glycan isolation procedure was validated with standard glycan mixtures and mass spectrometric analysis before and after purification step to ensure uniform glycan purification and quantitative detection of sialic acid residues in the analysis conditions. The method was optimized for glycan analysis in the used m/z range. For the quantitative glycan profile analyses, mass spectrometric raw data were cleaned by carefully removing the effect of isotopic pattern overlapping, multiple alkali metal adduct signals, products of elimination of water from the reducing oligosaccharides, and other interfering mass spectrometric signals not arising from the original glycans in the sample. The resulting cleaned profiles were normalized to 100% to allow comparison between samples.

### Preparation of Antibody Glycoforms: Normal and Hybrid-Type Glycoforms

CHO cell line DP-12 obtained from ATCC producing humanized IgG1 against IL-8 was cultured in normal conditions and with swainsonine. N-glycans were analyzed by mass spectrometric N-glycan profiling showing that the Fc domain N-glycans of the CHO cell supernatant-derived IgG were normal biantennary complex-type glycoform N-glycans with the major glycan signals at m/z 1485.6, 1647.6 and 1809.9 corresponding to the [M+Na]+ ions of Hex3HexNAc4dHex1, Hex4HexNAc4dHex1 and Hex5HexNAc4dHex1 oligosaccharides, respectively, while the IgG preparate produced with swainsonine was essentially completely (>99%) of the hybrid-type glycoform with the major (75% of total N-glycan signals) glycan signal at m/z 1768.7 corresponding to the [M+Na]+ ion of Hex6HexNAc3dHex1 oligosaccharide. The structure of the major product was the hybrid-type glycoform N-glycan Galβ4GlcNAcβ2Manα3[Manα3(Manα6)Manα6]Manβ4GlcNAcβ4(Fuc α6) GlcNAc based on sensitivity to β1,4-galactosidase (recombinant S. pneumoniae galactosidase, Glyko) digestion and known structure of the mannosidase II inhibition product. Other major Fc-domain N-glycan forms were Neu5Acα3Galβ4GlcNAcβ2Manα3[Manα3(Manα6)Manα6]Manβ4GlcNAcβ4(Fucα6)GlcNAc at m/z 2081.7 for the [M-H+2Na]+ ion (19%) according to mass spectrometric analysis and sensitivity to specific α2,3-sialidase (recombinant S. pneumoniae sialidase, Calbiochem) and GlcNAcβ2Manα3[Manα3(Manα6)Manα6]Manβ4GlcNAcβ4(Fucα6)GlcNAc at m/z 1606.6 (6%). In the hybrid-type glycoform no non-fucosylated N-glycans were detected.

### Monoantennary Glycoforms

A hybrid-type IgG glycoform preparate was subjected to Jack bean α-mannosidase (Sigma Aldrich) digestion in conditions similar to 50-65 U/ml enzyme for 2 days in 50 mM sodium acetate buffer pH 5.5 at +37° C. The products were purified by protein G affinity chromatography and N-glycan structures were analyzed as described above. The major glycan signal in the preparates was m/z 1444.5 corresponding to the [M+Na]+ ion of Hex4HexNAc3dHex1 oligosaccharide (70% of total N-glycan signals). The structure of the major product was the monoantennary glycoform N-glycan Galβ4GlcNAcβ2Manα3(Manα6)Manβ4GlcNAcβ4(Fucα6) GlcNAc based on sensitivity to β1,4-galactosidase digestion and known structure of the mannosidase II inhibition product. Other major Fc-domain N-glycan forms were Neu5Acα3Galβ4GlcNAcβ2Manα3(Manα6)Manβ4GlcNAcβ4(Fucα6)GlcNAc at m/z 2081.7 for the [M-H+2Na]+ ion (10%) according to mass spectrometric analysis and sensitivity to specific α2,3-sialidase (recombinant S. pneumoniae sialidase, Calbiochem) and GlcNAcβ2Manα3(Manα6) Manβ4GlcNAcβ4(Fucα6)GlcNAc at m/z 1606.6 (20%). Quantitative evaluation of the mass spectrum revealed that essentially all (>99%) of the detected N-glycan signals in the IgG preparates corresponded to these monoantennary glycoform structures and no non-fucosylated glycans were detected.

### Galactosylated and Sialylated Glycoforms

For galactosylation, antibodies were buffer-exchanged to 50 mM MOPS, pH 7.2, 20 mM MnCl2, using a NAP-5 column. 0.5 mU/μl of Calbiochem bovine milk β1,4-galactosyltransferase and 5 mM UDP-Gal was added to 6.25 mg/ml of antibody. Reactions were incubated overnight at +37° C. N-glycans were analyzed as described above. In typical reaction N-glycan galactosylation degree was increased to over 90% of N-glycans and in continued reactions N-glycan galactosylation degree was increased over 99% to essentially completely galactosylated forms. For subsequent α2,6-sialylation, 2.5 mU of Calbiochem α2,6-sialyltransferase, CMP-NeuNAc to 10 mM and BSA to 0.2 mg/ml were added to 100 μg protein (total volume of the reaction about 35 μl) and the reactions were incubated for about 42 h at +37° C. N-glycans were analyzed as described above. In a typical reaction N-glycan sialylation degree was increased to over 50% of N-glycans. In the α2,6-sialylated hybrid-type glycoform, the major N-glycan signal at m/z 2081.7 corresponding to Neu5Ac1Hex6HexNAc3dHex1 was 59% of the detected N-glycan signals while the other major N-glycan signal at m/z 1768.7 corresponding to Hex6HexNAc3dHex1 was 27% of the detected N-glycan signals (69% sialylation level of terminal galactose residues). In the α2,6-sialylated monoantennary glycoform, the major N-glycan signal at m/z 1757.7 corresponding to Neu5Ac1Hex4HexNAc3dHex1 was 54% of the detected N-glycan signals while the other major N-glycan signal was at m/z 1444.6 corresponding to Hex4HexNAc3dHex1. All the different antibody glycoforms were checked for structural integrity by protein G affinity chromatography as described above as well as polyacrylamide gel electrophoresis.

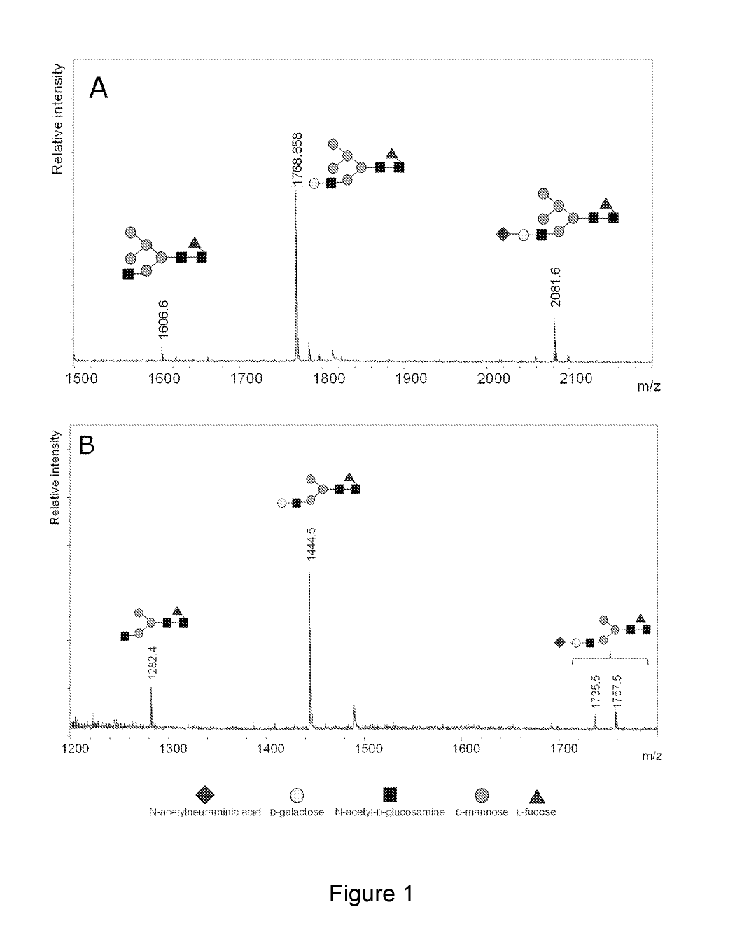

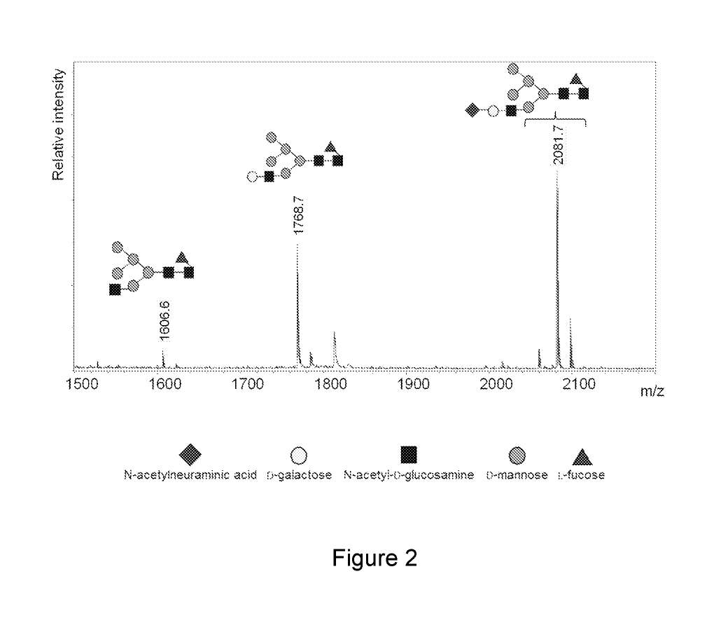

FIGS. 1 and 2 show exemplary mass spectra of hybrid-type and monoantennary glycoform N-glycans.

### Example 2

### Lectin Chromatography for Enrichment of Specific Glycoforms

α2,6-sialylated glycoforms of an anti-HER2 antibody were enriched by lectin affinity chromatography using Sambucus nigra lectin (SNA, Calbiochem) essentially as described in Stadlman et al. (Proteomics 9: 4143-4153, 2009) and Kaneko et al. (Science 313: 670-673, 2006). SNA was coupled 9 mg/ml to HiTrap NHS-activated HP 1 ml (GE Healthcare) according to manufacturer's instructions and the column was installed in Äkta Purifier HPLC system (GE Healthcare). α2,6-sialylated anti-HER2 antibody in Tris-buffered saline (TBS pH 7.4), 1 mM CaCl2, 1 mM MgCl2 (buffer A), was applied to SNA-affinity column equilibrated with buffer A at a flow rate of 0.2 ml/min. During sample injection the flow was stopped twice for 2 minutes. The unbound sample was washed from the column 0.4 ml/min with buffer A and the enriched sialylated antibodies were eluted 0.4 ml/min with TBS, 0.5 M lactose (buffer B).

### Example 3

### TNF-α Production Assay

TNF-α production assay was done essentially as described in Roda, J. M. et al. (The Journal of Immunology (2006), 177: 120-129). In short, wells of a 96-well flat-bottom plate were coated with glycoform antibodies 50, 100 or 200 μg/ml in PBS o/n at 4° C. and washed with cold PBS and warm RPMI-1640 medium. Peripheral blood mononuclear cells (PBMC) were isolated from healthy volunteers using Vacutainer CPT tubes (BD), washed with PBS and RPMI-1640 medium and suspended 106 cells/ml in medium supplemented with 10% fetal calf serum, 100 U/ml penicillin, 100 μg/ml streptomycin and glutamine. PBMC were added to antibody coated wells 2×105 cells/well and the plates were incubated o/n 37° C. in humidified atmosphere and 5% CO2. TNF-α production was analyzed from cell culture supernatants using Human TNF-α Immunoassay kit (R&D Systems).

The potencies of the normal IgG and hybrid-type antibody glycoforms to induce TNF-α production and thus mediate FcγR-dependent cellular cytotoxicity (Roda et al. 2006) were analyzed and found to be at the same level.

### Example 4

### Receptor Binding Assays

### Printing of Arrays.

Arrays were printed onto Schott Nexterion H MPX-16 slides (Schott Technical Glass Solutions GmbH, Jena, Germany). Antibody isoform and control protein samples were diluted to 0.5 mg/ml with a buffer that had been made by bringing 100 mM sodium citrate buffer pH 2.6 to pH 7 by adding 1 M Na2HPO4. The samples were printed at a volume of ˜400 pL per spot using a Scienion sciFLEXARRAYER S5 non-contact printer (Scienion AG, Berlin, Germany). For each sample concentration, 6 replicates were printed. 6 replicate spots of Cy3-labeled protein served as positive control and 6 replicate spots of printing buffer solution served as negative controls. In the arrays the distance between adjacent spots was approximately 380 μm. Arrays of up to 24 different isoforms and control substances were printed resulting in 144 spots/array. The printed array slides were incubated in 75% humidity in room temperature overnight, allowed to dry in room temperature and stored until use in −20° C. in a desiccator.

Hybridization with Effector Molecules and Reading of Arrays

### Preparation of Binding Proteins for Assays.

Recombinant human DC-SIGN receptor was from R&D Systems Inc. (USA) and C1q complement was from Quidel (San Diego, Calif., USA). These binding proteins were labeled with NHS-activated Cy3 or Cy5 (GE Healthcare, UK) according to manufacturer's instructions and purified from excess reagent by changing the buffer to phosphate buffered saline (PBS) in NAP-5 columns (GE Healthcare, UK).

### Assay Procedure to Evaluate DC-SIGN and C1q Binding Affinities.

Printed slides were blocked with 25 mM ethanolamine in 100 mM borate buffer, pH 8.5 for at least one hour in room temperature. Slides were rinsed three times with PBS-Tween (0.05% Tween), once with PBS and once with water. A Schott Nexterion MPX superstructure (Schott Technical Glass Solutions GmbH, Jena, Germany) was attached to create wells. Arrays were incubated with various concentrations of labeled binding proteins in 60 μl volume of PBS buffer. In addition, 1 mM CaCl2 was added to DC-SIGN incubations. Incubations were carried out for 2.5 h at room temperature, after which the slides were washed five times in PBS-Tween, once with PBS, rinsed with water and dried using nitrogen gas stream. Arrays were imaged using Tecan's LS Reloaded laser scanner (Tecan Group Ltd., Switzerland) at excitation wavelengths of 532 and 633 nm and detection wavelengths of 575 and 692 nm for Cy3 and Cy5, respectively. The images were quantified using Array Pro software.

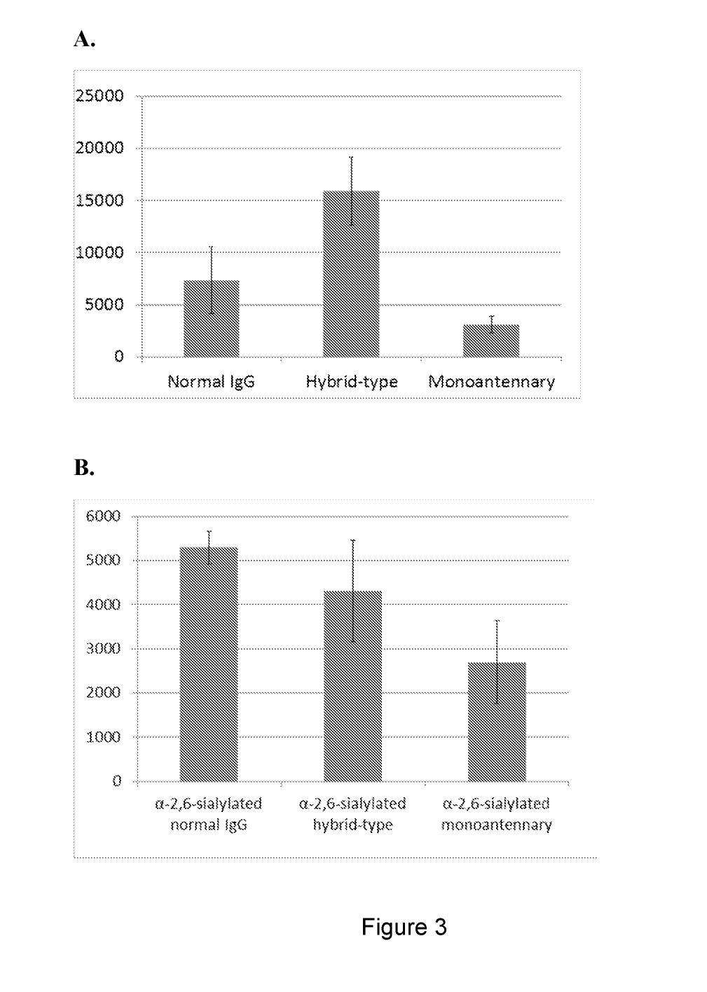

Results of a typical DC-SIGN binding assay are shown in FIGS. 3 A and B. The relative affinities of non-α2,6-sialylated antibody glycoforms to DC-SIGN were in the following order (FIG. 3A): hybrid-type > normal IgG> monoantennary; while the relative affinities of α2,6-sialylated antibody glycoforms to DC-SIGN were in the following order (FIG. 3B): α2,6-sialylated normal IgG=α2,6-sialylated hybrid-type >α2,6-sialylated monoantennary.

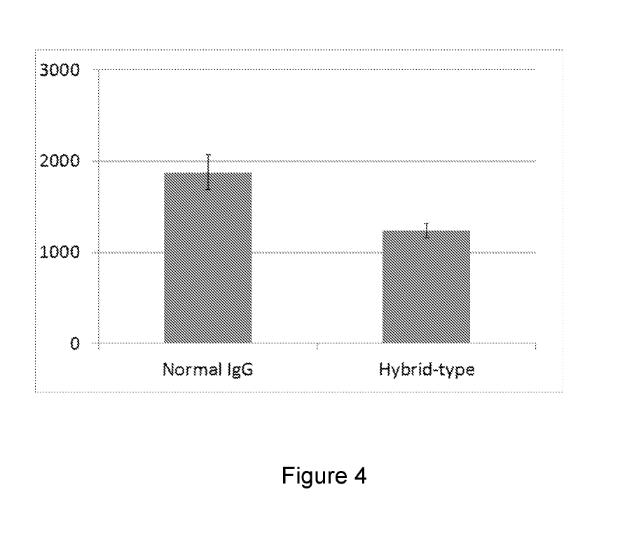

Results of a typical C1q-binding assay are shown in FIG. 4. The relative affinities of the antibody glycoforms to C1q were in the following order: monoantennary > normal IgG> hybrid-type.

### Example 5

### Inhibition of Glycosylation Enzymes with Specific siRNAs in HEK-293 Cells

Glycosylation targeted siRNA probes were obtained from Qiagen. Human embryonal kidney HEK-293 cells were cultured in 384-well plates in standard culture conditions and transfected for 48 h with each siRNA in eight replicate experiments. After the transfection, cells were fixed and permeabilized, labelled with lectins PHA-L and AAL fluorescent-labelled with Cy3 as described above and the amount of label was quantitated by image acquisition and analysis with Olympus scanR system.

One of the anti-MGAT siRNAs, SI04314219, inhibited branched complex-type N-glycan biosynthesis as judged by decreased labeling with PHA-L (labeling intensity fold change −0.66). This indicated that this siRNA had decreased the activity of GnTII in these cells, leading to increased amounts of monoantennary N-glycans.

Three of the anti-MAN2A1 siRNAs, SI00036729, SI00036722 and SI00036743, inhibited branched complex-type N-glycan biosynthesis as judged by decreased labeling with PHA-L (labeling intensity fold changes −0.20, −0.58 and −0.81, respectively). This indicated that these siRNAs had decreased the activity of mannosidase II in these cells, leading to increased amounts of hybrid-type N-glycans.

One of the anti-MAN2A2 siRNAs, SI00084679, inhibited branched complex-type N-glycan biosynthesis as judged by decreased labeling with PHA-L (labeling intensity fold change −0.34) and increased fucosylation as judged by increased labeling with AAL (labeling intensity fold change 0.37). This indicated that these siRNAs had decreased the activity of mannosidase IIx in these cells, leading to increased amounts of core-fucosylated hybrid-type N-glycans.

The utilized siRNA probes are identified by Qiagen SI codes as shown in Table 1.

TABLE 1

| Gene | Enzyme | Qiagen SI codes |
|---|---|---|
| MGAT2 | GnTII | SI04248286, SI04308521, |
| | | SI04314219, SI00630987 |
| MAN2A1 | mannosidase | SI00036729, SI00036722, |
| | II | SI00036743, SI00036736 |
| MAN2A2 | mannosidase | SI00084672, SI00084679, |
| | IIx | SI00084658, SI00084665 |

### Example 6

### In Vivo Half-Life of Humanized Antibody Glycoforms

The purpose of the study was to measure in vivo serum biodistribution of anti-IL-8 IgG1 humanized antibody glycoforms in healthy mice after a single i.v. administered dose of antibody. The test animals were female FVB/N mice. Background serum samples (100 μl blood) were taken from all animals three days before the start of the experiment. Serum samples were obtained in serum isolation tubes by centrifuging the blood samples. 50 μg of antibody was injected i.v. via the tail vein in 110 μl phosphate-buffered saline at start of day 1 of the experiment. 100 μl blood samples were taken from all animals about 10 min after dosing of test substances and on days 2, 3, 5, 8 and 15. The test substances contained 0.45 g/1 anti-IL-8 antibody glycoforms in sterile-filtered phosphate-buffered saline. 100 μl blood samples were collected and serum was isolated. The rates of elimination from serum of both complex-type CHO-expressed anti-IL-8 IgG1 humanized antibody and its hybrid-type glycoform were essentially similar in mice: when 50 μg effective dose was administered at day 1, at day 15 the remaining serum concentration of both antibody forms was between 1 μg/ml and 2 μg/ml.

N-glycans were isolated and analysed by MALDI-TOF mass spectrometry as described above from the antibody before administration to animals, showing that the major Fc domain N-glycan structures were core-fucosylated hybrid-type N-glycans of the structures [(Neu5Ac)₀₋₁Galβ4]₀₋₁GlcNAcβ2Manα3[Manα3(Manα6)Manα6]Manβ4GlcNAcβ4(Fucα6)GlcNAc (over 90% of the total N-glycans).

### Example 7

### In Vitro Production of Trastuzumab Glycoforms

Trastuzumab (Genentech/Roche) was galactosylated with bovine milk β1,4-galactosyltransferase (Sigma-Aldrich) and sialylated with human recombinant ST6GAL1 α2,6-sialyltransferase (R&D Systems) as described in the preceding examples. N-glycans were analysed by MALDI-TOF mass spectrometry as described above, showing that the Fc domain N-glycans were essentially completely galactosylated and the major N-glycans were the signals at m/z 2122.7 (over 50% of the glycan signal intensity) corresponding to the monosialylated and fully galactosylated N-glycan Neu5Ac1Hex5HexNAc4dHex1 and at m/z 1809.6 (over 35% of the glycan signal intensity) corresponding to the fully galactosylated N-glycan Hex5HexNAc4dHex1. The sialic acid was located at the α1,3-branch of the N-glycan due to the branch specificity of the ST6GAL1 enzyme. The antibody preparate was further processed by enzymatic digestion at +37 C for 1 day by β1,4-galactosidase (recombinant S. pneumoniae galactosidase, Glyko) and β-glucosaminidase (recombinant S. pneumoniae glucosaminidase) after buffer exchange into 50 mM sodium acetate pH 5.5, to remove the non-sialylated antennae. The preparate was then exchanged into buffer A and chromatographed on Sambucus nigra lectin column as described above to recover the α2,6-sialylated monoantennary trastuzumab glycoform. N-glycans were analysed by MALDI-TOF mass spectrometry after sialidase A digestion (Glyko), showing that the major Fc domain N-glycan structure in the α2,6-sialylated monoantennary trastuzumab glycoform was the monosialylated and core-fucosylated monoantennary N-glycan Neu5Acα6Galβ4GlcNAcβ2Manα3(Manα6)Manβ4GlcNAcβ4(Fucα6)GlcNAc (67% of the total N-glycans) as evidenced by the detected desilylated glycan signal at m/z 1444.5 corresponding to Hex4HexNAc3dHex1.

### Example 8

### Production of Trastuzumab Glycoforms in CHO Cells

Trastuzumab was produced transiently in CHO-S cells with FreeStyle™ Max Expression System (Life Technologies) according to manufacturer's instructions. The trastuzumab amino acid sequences were according to the IMGT database (http://www.imgt.org) for the light chain (7637_L) and heavy chain (7367_H) sequences. Optimized nucleotide sequences encoding the heavy and light chain sequences with functional signal sequences were purchased from GeneArt (Life Technologies) and cloned separately into pCEP4 expression vectors (Life Technologies). For antibody expression, the FreeStyle™ CHO-S cells were transfected 1:1 with light chain and heavy chain vectors.

For production of hybrid-type trastuzumab glycoforms, the transfected cells were incubated with swainsonine as described in the preceding examples. N-glycosidase liberated N-glycans were analysed by MALDI-TOF mass spectrometry from protein G purified antibody as described above. The major N-glycan signals corresponded to the core-fucosylated hybrid-type N-glycans Hex5HexNAc3dHex1, Hex6HexNAc3dHex1 and NeuAc1Hex6HexNAc3dHex1; corresponding to the N-glycan structures GlcNAcβ2Manα3[Manα3(Manα6)Manα6]Manβ4GlcNAcβ4(Fucα6)GlcNAcβ4GlcNAcβ2Manα3[Manα3(Manα6)Manα6]Manβ4GlcNAcβ4(Fucα6) GlcNAc and Neu5Acα3Galβ4GlcNAcβ2Manα3[Manα3(Manα6)Manα6]Manβ4GlcNAcβ4(Fucα6) GlcNAc.

For production of monoantennary trastuzumab glycoforms, the transfected cells were incubated with swainsonine and digested with α-mannosidase as described above. N-glycosidase liberated N-glycans were analysed by MALDI-TOF mass spectrometry from protein G purified antibody as described above. The major N-glycan signals corresponded to the core-fucosylated monoantennary N-glycans Hex3HexNAc3dHex1, Hex4HexNAc3dHex1 and NeuAc1Hex4HexNAc3dHex1; corresponding to the N-glycan structures GlcNAcβ2Manα3(Manα6)Manβ4GlcNAcβ4(Fucα6)GlcNAc, Galβ4GlcNAcβ2Manα3(Manα6)Manβ4GlcNAcβ4(Fucα6)GlcNAc and Neu5Acα3Galβ4GlcNAcβ2Manα3(Manα6)Manβ4GlcNAcβ4(Fucα6)GlcNAc.

As is clear for a person skilled in the art, the invention is not limited to the examples and embodiments described above, but the embodiments can freely vary within the scope of the claims.

1-26. (canceled)

27. A pharmaceutical composition comprising a glycoprotein comprising the Fc domain of an antibody, or a fragment thereof, comprising an Asn residue and an oligosaccharide structure attached thereto, characterised in that the oligosaccharide structure has a structure according to formula I 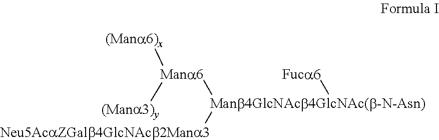
wherein
(β-N-Asn)=β-N linkage to Asn;
Z=3 or 6;
x=0 or 1; and y=0 or 1;
wherein at least 10% of the oligosaccharide structures attached to glycoproteins in the composition consist of oligosaccharide structures according to formula I.

28. The pharmaceutical composition according to claim 27, wherein at least 50%, or at least 66.7%, or at least 80%, or at least 90% of the oligosaccharide structures attached to glycoproteins in the composition consist of oligosaccharide structures according to formula I.

29. The pharmaceutical composition according to claim 27, wherein the oligosaccharide structure has the structure according to formula I wherein x=1 and y=1.

30. The pharmaceutical composition according to any claim 27, wherein the Fc domain is a human Fc domain.

31. The pharmaceutical composition according to claim 27, wherein the glycoprotein is a fusion protein comprising an Fc domain.

32. The pharmaceutical composition according to claim 27, wherein the glycoprotein is a human antibody, a humanized antibody or a chimeric antibody comprising a human Fc domain.

33. The pharmaceutical composition according to claim 32, wherein the glycoprotein is an IgG antibody.

34. The pharmaceutical composition according to claim 27, wherein at least 95%, 98%, 99%, 99.5%, 99.8%, 99.9% or essentially all of the oligosaccharide structures attached to the glycoproteins in the composition comprise the Fuc residue.

35. A pharmaceutical composition comprising a glycoprotein comprising the Fc domain of an antibody, or a fragment thereof, comprising an Asn residue and an oligosaccharide structure attached thereto, wherein at least 66.7%, or at least 80%, or at least 90%, or at least 95%, or at least 98%, or at least 99%, or at least 99.5%, or essentially all of the oligosaccharide structures attached to glycoprotein in the composition consist of oligosaccharide structures according to formula II 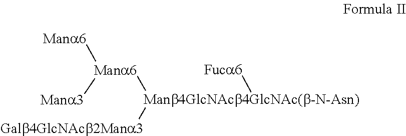
wherein
(β-N-Asn)=β-N linkage to Asn.

36. The pharmaceutical composition according to claim 27, wherein the composition further comprises a glycoprotein comprising the Fc domain of an antibody, or a fragment thereof, comprising an Asn residue and an oligosaccharide structure attached thereto, wherein the oligosaccharide structure has a structure according to formula III 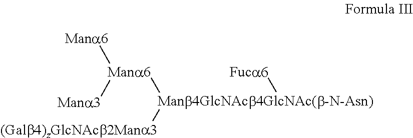
wherein
(β-N-Asn)=β-N linkage to Asn;
z=0 or 1; and
wherein at least 10% of the oligosaccharide structures attached to glycoprotein in the composition consist of oligosaccharide structures according to formula III.

37. The pharmaceutical composition according to claim 36, wherein at least 50%, 60%, 70%, 80%, 90%, 95%, 98%, 99%, 99.5% or essentially all of the oligosaccharide structures attached to glycoprotein in the composition consist of oligosaccharide structures according to formula I and of oligosaccharide structures according to formula III.

38. The pharmaceutical composition according to claim 27, wherein the glycoprotein is an antibody directed against human vascular endothelial growth factor (VEGF), epidermal growth factor receptor 1 (EGFR), tumor necrosis factor alpha (TNF-α), CD20, epidermal growth factor receptor 2 (HER2/neu), CD52, CD33, CD11a, glycoprotein CD25, IgE, IL-2 receptor, or respiratory syncytial virus (RSV).

39. The pharmaceutical composition according to claim 27, wherein the antibody is bevacizumab, tositumomab, etanercept, trastuzumab, Adalimumab, alemtuzumab, gemtuzumab ozogamicin, efalizumumab, rituximab, infliximab, abciximab, baasiliximab, palivizumab, omalizumab, daclizumab, cetuximab, panitumumab, or ibritumomab tiuxetan.

40. The pharmaceutical composition according to claim 35, wherein the composition further comprises a glycoprotein comprising the Fc domain of an antibody, or a fragment thereof, comprising an Asn residue and an oligosaccharide structure attached thereto, wherein the oligosaccharide structure has a structure according to formula III 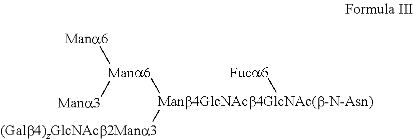
wherein
(β-N-Asn)=β-N linkage to Asn;
z=0 or 1; and
wherein at least 10% of the oligosaccharide structures attached to glycoprotein in the composition consist of oligosaccharide structures according to formula III.

41. The pharmaceutical composition according to claim 40, wherein at least 50%, 60%, 70%, 80%, 90%, 95%, 98%, 99%, 99.5% or essentially all of the oligosaccharide structures attached to glycoprotein in the composition consist of oligosaccharide structures according to formula I and of oligosaccharide structures according to formula III.

42. The pharmaceutical composition according to claim 35, wherein the glycoprotein is an antibody directed against human vascular endothelial growth factor (VEGF), epidermal growth factor receptor 1 (EGFR), tumor necrosis factor alpha (TNF-α), CD20, epidermal growth factor receptor 2 (HER2/neu), CD52, CD33, CD 11a, glycoprotein IIb/IIIa, CD25, IgE, IL-2 receptor, or respiratory syncytial virus (RSV).

43. The pharmaceutical composition according to claim 35, wherein the antibody is bevacizumab, tositumomab, etanercept, trastuzumab, Adalimumab, alemtuzumab, gemtuzumab ozogamicin, efalizumumab, rituximab, infliximab, abciximab, baasiliximab, palivizumab, omalizumab, daclizumab, cetuximab, panitumumab, or ibritumomab tiuxetan.

44. A host cell comprising a polynucleotide encoding the protein moiety of a glycoprotein defined in claim 27, wherein said host cell has
a) reduced activity of mannosidase II or GnTII, and
b) optimized, or increased, activity of β4-galactosyltransferase and/or α2,3/6-sialyltransferase
compared to the parent cell.

45. The host cell according to claim 44, wherein said host cell has increased activity of α2,6-sialyltransferase compared to the parent cell.

46. The host cell according to claim 44, wherein said host cell further has increased activity of core fucosylation compared to the parent cell.

47. The host cell according to claim 44, wherein said host cell further has decreased activity of a sialidase compared to the parent cell.

48. A method of treating autoimmune diseases, inflammatory disorders or any other disease where binding to an antibody target or increased anti-inflammatory activity with reduced cytotoxic activity is desired, wherein the composition according to claim 27 is administered to a human or animal in an effective amount.

49. A method for producing the composition according to claim 27, wherein it comprises the steps of
a) culturing a host cell comprising a polynucleotide encoding the protein moiety of a glycoprotein in the presence of mannosidase II inhibitor; or the steps of
a′) culturing a host cell, wherein the host cell has
i) reduced activity of mannosidase II or GnTII, and
ii) optimized, or increased, activity of β4-galactosyltransferase and/or α2,3/6-sialyltransferase compared to the parent cell; and
a″) recovering the glycoprotein composition from the host cell culture.

50. The method according to claim 49, wherein it further comprises the steps of
b) contacting the product of step a), a′), or a″) with an β1,4-galactosyltransferase in the presence of UDP-Gal; and/or
c) contacting the product of step b) with a α2,6-sialyltransferase in the presence of CMP-NeuNAc, and/or contacting the product of the previous step with an α-mannosidase and/or recovering the glycoprotein composition, and adding a pharmaceutically acceptable carrier.

51. A method for producing the composition according to claim 35, wherein it comprises the steps of
a) culturing a host cell comprising a polynucleotide encoding the protein moiety of a glycoprotein wherein the glycoprotein consists of oligosaccharide structures according to formula II 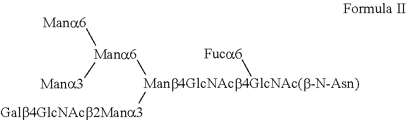
wherein
(β-N-Asn)=β-N linkage to Asn in the presence of mannosidase II inhibitor; or the steps of
a′) culturing a host cell, wherein the host cell has
i) reduced activity of mannosidase II or GnTII, and
ii) optimized, or increased, activity of β4-galactosyltransferase and/or α2,3/6-sialyltransferase compared to the parent cell; and
a″) recovering the glycoprotein composition from the host cell culture.

52. The method according to claim 25, wherein it further comprises the steps of
b) contacting the product of step a), a′), or a″) with an β1,4-galactosyltransferase in the presence of UDP-Gal; and/or
c) contacting the product of step b) with a α2,6-sialyltransferase in the presence of CMP-NeuNAc, and/or contacting the product of the previous step with an α-mannosidase and/or recovering the glycoprotein composition, and adding a pharmaceutically acceptable carrier.
